(19) United States
(12) Patent Application Publication
(10) Pub. No.: US 2026/0158059 A1
(43) Pub. Date: Jun. 11, 2026

(54) READY-TO-USE SODIUM BICARBONATE FORMULATIONS AND PRODUCTS

(71) Applicant: INFORLIFE SA, CAMPASCIO (CH)

(72) Inventors: Sergio Dusci, Tresivio (IT); Abramo Civera, Piateda (IT)

(73) Assignee: INFORLIFE SA, CAMPASCIO (CH)

(21) Appl. No.: 18/970,662

(22) Filed: Dec. 5, 2024

(51) Int. Cl. A61K 33/10; A61J 1/10; A61J 1/14

(52) U.S. Cl. CPC A61K 33/10; A61J 1/10; A61J 1/1468

(57) ABSTRACT

The invention includes a sodium bicarbonate product that is sterile and ready-to-use and can be stored at room temperature for a period of six months or more, including an aqueous sodium bicarbonate solution at a concentration suitable for administration to a patient, which does not contain a preservative or antimicrobial, in an infusion container. The invention includes methods of making and using the sodium bicarbonate product.

### TECHNICAL FIELD

The present invention relates to ready-to-use, sterile, premixed formulations of Sodium Bicarbonate that are stable for 6 months or longer, including up to 24 months or longer in an infusion container when stored at room temperature. The present invention also relates to methods of treating diseases or disorders characterized by metabolic acidosis or low serum bicarbonate levels.

### BACKGROUND

Sodium Bicarbonate is a systemic alkalinizing agent which, when given intravenously, will increase plasma bicarbonate, buffer excess hydrogen ion concentration, raise blood pH and reverse the clinical manifestations of acidosis. A normal blood pH range is from 7.35 to 7.45. Metabolic acidosis is characterized by an increase in the hydrogen ion concentration in the blood plasma, which reduces blood pH and results in an abnormally low serum bicarbonate level. Metabolic acidosis can be caused by a variety of causes including diabetic ketoacidosis, lactic acidosis, septic shock, and chronic kidney dysfunction that produces an excess net dietary acid load.

Sodium bicarbonate dissociates in water to provide sodium and bicarbonate ions (HCO3-). Sodium is the principal cation of the extracellular fluid and plays a large part in the therapy of fluid and electrolyte disturbances. Bicarbonate is a normal constituent of body fluids and the normal plasma level ranges from 24 to 31 mmol/L. Aqueous sodium bicarbonate solutions are unstable, as sodium bicarbonate tends to hydrolyze to form carbon dioxide. The decomposition of sodium bicarbonate leads to an increase in the pH of the solution.

Most preparations of sodium bicarbonate are commercially available as concentrated 0.5 mEq/mL, 0.9 mEq/mL and 1 mEq/mL sodium bicarbonate solutions in a 10-50 mg/ml vial, and must be diluted and transferred to an infusion container shortly before administration to a patient. The container of a product intended for parenteral use has to ensure continuous compliance with storage and handling specifications throughout the product shelf life to maintain functionality and drug delivery accuracy. Several factors have to be considered when choosing the right packaging/container for an injectable product, such as drug product formulation properties, dosage, type of application, stability, storage conditions and duration, and end-user friendliness.

There is a need for a sterile, ready-to-use infusion container comprising a stable, liquid formulation of sodium bicarbonate which can be administered to a patient in need thereof without dilution, deviation, or manipulations and while preserving the sterility of the product, thereby avoiding a compromise with the sterility, an error in dosing accuracy and/or in medicament preparation, etc. There is a need for a ready-to-use sodium bicarbonate solution that has a shelf-life of 12 months or more at room temperature. There is a need for a sodium bicarbonate solution in a ready-to-use infusion bag that can be stored for 6 months, 9 months, 12 months, 24 months or longer at room temperature without unacceptable product degradation or variation in pH. There is a need for a ready-to-use sodium bicarbonate solution that does not show significant variation in pH during the infusion process.

### SUMMARY OF THE INVENTION

In the present invention, the inventors have overcome multiple problems of prior sodium bicarbonate formulations, such as additional costs and inconvenience, the risk of contamination due to inadvertent medical error, and the risk of overdosing or underdosing patients, by providing a sodium bicarbonate product capable of long-term storage in the form of a ready-to-use product containing a stable, sterile sodium bicarbonate solution suitable for infusion directly into a subject. The inventors have developed a ready-to-use, stable, sterile, formulation of sodium bicarbonate in an infusion container. The inventors have developed a ready-to-use, stable, sterile, formulation of sodium bicarbonate at ready-to-use concentrations that does not require a preservative, chelating agent, antioxidant, or antimicrobials. In one aspect, the ready-to-use, stable, sterile, formulation of sodium bicarbonate of the disclosure does not contain bisulfite or other sulfite-containing preservatives. The ready-to-use, stable, sterile, formulation of sodium bicarbonate in an infusion container may further be packaged with an overwrap film that does not require vacuum sealing and may contain air between the infusion container and the overwrap film.

For materials being considered for use as primary packing materials for the infusion container for the ready-to-use sodium bicarbonate solution of the present disclosure, the challenge has been to develop packaging that is compatible with the contents, minimizes extractables/leachables, minimizes gas permeability, is solvent-resistant, and is durable. The packaging of a pharmaceutical product must be compatible with the specific drug and its specific formulation components, and desirably, the packaging and drug formulation should be stable and mutually compatible under aseptic conditions, as packaging materials can affect the condition of different formulations in unpredictable ways. The inventors have beneficially developed a ready-to-use sodium bicarbonate formulation in an infusion bag that is shelf-stable in an infusion container without the need to vacuum seal the overwrap over the infusion container and is sterile.

The present invention relates to premixed pharmaceutical compositions of sodium bicarbonate. The premixed pharmaceutical compositions of sodium bicarbonate of the present invention are formulated for administration to a patient, without the need to reconstitute or dilute the composition prior to administration, and without the need for terminal sterilization, without the need to vacuum seal the overwrap over the infusion container, and without use of preservatives and antimicrobials.

In certain aspects, the sodium bicarbonate product of the present disclosure comprises an aqueous sodium bicarbonate solution comprising about 0.1 to 0.2 mEq/mL sodium bicarbonate, a tonicity agent, and water for injection, wherein the sodium bicarbonate solution is packaged in an infusion container and the infusion container is further packaged in an overwrap film comprising aluminum, wherein the sodium bicarbonate product is sterile and ready-to-use and has a shelf-life of at least 6 months when stored at a controlled room temperature of 25° C.±2° C. with relative humidity (RH) at 40%±5%.

In one aspect, the sodium bicarbonate product of the present disclosure comprises an aqueous sodium bicarbonate solution comprising sodium bicarbonate, a tonicity agent, and water for injection, wherein the sodium bicarbonate product is sterile and ready-to-use, and wherein the sodium bicarbonate solution is contained in an infusion bag, wherein the infusion bag comprises a flexible multilayer film wherein at least one layer comprises a polymer selected from the group consisting of maleic anhydride grafted polyethylene and poly(ethylene-co-1-octene).

In one aspect, the sodium bicarbonate product of the present disclosure comprises an aqueous sodium bicarbonate solution comprising about 0.1 to 0.2 mEq/mL sodium bicarbonate, a tonicity agent, and water for injection, wherein the sodium bicarbonate product is sterile and ready-to-use, and wherein the sodium bicarbonate solution is contained in an infusion bag, wherein the infusion bag comprises a flexible multilayer film wherein at least one layer comprises poly(ethylene-co-1-octene) and/or maleic anhydride, wherein the sodium bicarbonate solution is substantially free of preservatives, substantially free of antimicrobials, or substantially free of both preservatives and antimicrobials, wherein the sodium bicarbonate solution is stable for at least 12 months when stored at a controlled room temperature of 25° C.±2° C. with relative humidity (RH) at 40%±5%.

In some aspects, the pH of the aqueous sodium bicarbonate solution is maintained within a range of 7.0 to 8.5 following storage for 6 months at a controlled room temperature of 25° C.±2° C. with relative humidity (RH) at 40%±5%. In some aspects, the pH of the aqueous sodium bicarbonate solution is maintained within a range of 7.5 to 8.5 following storage for 6 months at a controlled room temperature of 25° C.±2° C. with relative humidity (RH) at 40%±5%.

In one aspect, present invention relates to a method for treating metabolic acidosis in a patient in need thereof, comprising administering an aqueous sodium bicarbonate solution comprising 0.1 mEq/mL or 0.15 mEq/mL sodium bicarbonate from a sterile, ready-to-use infusion bag, wherein the pH of the sodium bicarbonate solution changes less than 0.1 pH units, or less than 0.2 pH units, during the infusion time. In some aspects, the entire contents of a sterile, ready-to-use infusion bag comprising 0.1 mEq/mL or 0.15 mEq/mL sodium bicarbonate solution is administered to the patient, and the pH of the sodium bicarbonate solution changes less than 0.1 pH units, or less than 0.2 pH units, during the infusion time. In some aspects, the sterile, ready-to-use infusion bag comprising 0.1 mEq/mL or 0.15 mEq/mL sodium bicarbonate is stored at room temperature for three months or more prior to administration of the solution to the patient.

In one aspect, present invention relates to a method for treating metabolic acidosis in a patient in need thereof, comprising administering a volume of from about 200 mL to about 1 liter (1 L) of an aqueous sodium bicarbonate solution comprising 0.1 mEq/mL or 0.15 mEq/mL sodium bicarbonate from a sterile, ready-to-use infusion bag to the patient over a time period of about 4 to 8 hours, wherein the pH of the sodium bicarbonate solution changes less than 0.1 pH units, or less than 0.2 pH units, during the infusion time.

In one aspect, present invention relates to a method for treating metabolic acidosis in a patient in need thereof, comprising administering a volume of from about 200 mL to about 1 liter (1 L) of an aqueous sodium bicarbonate solution comprising 0.1 mEq/mL or 0.15 mEq/mL sodium bicarbonate from a sterile, ready-to-use infusion bag to the patient over a time period of about 12 to 24 hours, wherein the pH of the sodium bicarbonate solution changes less than 0.1 pH units, or less than 0.2 pH units, during the infusion time.

In some aspects, the sodium bicarbonate solution is aseptically filled into the infusion container in the presence of carbon dioxide. In some aspects, the sodium bicarbonate solution is filled into the infusion container such that headspace is minimized or eliminated. For example, the sodium bicarbonate solution may be filled into the infusion container such that headspace occupies less than 5% of the full container volume.

### BRIEF DESCRIPTION OF THE DRAWINGS

FIG. 1 shows a graph comparing pH measurements of batches of 12.6 mg/mL sodium bicarbonate solutions of the present disclosure filled in a glass container under different conditions over a 300-minute period, the different conditions comprising varying correction of pH with carbon dioxide and the amount of headspace (“HS”). “A” shows pH measurements of batch RD004-23 with no correction of pH with CO₂ and 50% HS; “B” shows pH measurements of batch RD003-23 with no correction of pH with CO₂ and no HS; “C” shows pH measurements of batch RD006-23 with correction of pH with CO₂ and 50% HS; “D” shows pH measurements of batch RD007-23 with correction of pH with CO₂ and 50% HS saturated with CO₂; and “E” shows pH measurements of batch RD005-23 with correction of pH with CO₂ and no HS.

FIG. 2 shows a graph comparing pH measurements of batches of 12.6 mg/mL sodium bicarbonate solutions of the present disclosure stored over a 24-hour period in three different infusion bags (Renolit MF518H, Nexcel M312A and Renolit Solmed Infuflex 9101 infusion bags) with and without pH correction using carbon dioxide. “A” shows pH measurements of batch RD004-23 stored in Renolit MF518H with no pH correction; “B” shows pH measurements of batch RD005-23 stored in Renolit MF518H and with initial pH correction to pH 7.0; “C” shows pH measurements of batch RD006-23 stored in Nexcel M312A and with initial pH correction to pH 7.0; “D” shows pH measurements of batch RD008-23A stored in Renolit Solmed Infuflex 9101 with no pH correction; and “E” shows pH measurements of batch RD008-23B stored in Renolit Solmed Infuflex 9101 with initial pH correction to pH 7.0.

FIG. 3 shows a graph comparing pH measurements over a 24-hour period for batches of 12.6 mg/mL sodium bicarbonate solutions of the present disclosure stored in different primary packaging (Renolit MF518H or Nexcel M312A infusion bags) with and without an overwrap and with and without initial pH correction to pH 7.0 with carbon dioxide. “A” shows pH measurements of batch RD004-23 stored in Renolit MF518H without an overwrap and with no pH correction; “B” shows pH measurements of batch RD004-23 stored in Renolit MF518H with an overwrap and with no pH correction; “C” shows pH measurements of batch RD005-23 stored in Renolit MF518H without an overwrap and with a pH correction; “D” shows pH measurements of batch RD005-23 stored in Renolit MF518H with an overwrap and with pH correction; “E” shows RD006-23 stored in Nexcel M312A without an overwrap and with pH correction; and “F” shows RD006-23 stored in Nexcel M312A with an overwrap and with pH correction.

FIG. 4 shows pH measurements over a 160-hour period of batches of 12.6 mg/ml sodium bicarbonate solution of the present disclosure stored in a Renolit Solmed Infuflex 9101 infusion bag with and without pH correction and headspace (“HS”). “A” shows pH measurements of batch RD008-23 stored in Renolit Solmed Infuflex 9101 with no pH correction and no HS; “B” shows pH measurements of batch RD008-23C stored in Renolit Solmed Infuflex with pH correction and with HS; and “C” shows RD008-23B with Renolit Solmed Infuflex 9101 with pH correction and no HS.

FIG. 5 shows a graph comparing pH measurements over a 24-hour period of batches of 12.6 mg/mL sodium bicarbonate solution of the disclosure stored in a Renolit Solmed Infuflex 9101 infusion bag with an infusion set and with and without headspace. “A” shows pH measurements of batch RD008-23E stored in Renolit Solmed Infuflex 9101 with an infusion set and HS; and “B” shows pH measurements of batch RD008-23D stored in Renolit Solmed Infuflex 9101 with an infusion set and no HS.

FIG. 6 shows a schematic of the change in ratio of headspace volume to drug product volume during infusion at the start of infusion (t=0) to 6\hat{ }Time station, wherein each time station corresponds to: 10 minutes of continuous infusion for a total of 1 hour of infusion time; 1 hour of infusion for a total of 8 hours of infusion time; and 4 hours of infusion for a total of 24 hours of infusion time.

FIG. 7 shows a graph comparing pH measurements over a 60-minute period of infusion for batches of the ready-to-use, aseptically filled 12.6 mg/mL sodium bicarbonate product of the present disclosure at pH targets of pH 7.8 (3R032), pH 7.5 (3R033) and pH 7.2 (3R034).

FIG. 8 shows a graph comparing pH measurements over an 8-hour period of infusion for batches of the ready-to-use, aseptically filled 12.6 mg/mL sodium bicarbonate product of the present disclosure at pH targets of pH 7.8 (3R032), pH 7.5 (3R033) and pH 7.2 (3R034).

FIG. 9 shows a graph comparing pH measurements over a 24-hour period of infusion of the ready-to-use, aseptically filled 12.6 mg/mL sodium bicarbonate product of the present disclosure at pH targets of pH 7.8 (3R032), pH 7.5 (3R033) and pH 7.2 (3R034).

FIG. 10 shows a graph illustrating the assay of sodium bicarbonate expressed as a percentage of the target concentration of 12.6 mg/mL sodium bicarbonate, in solutions with pH targets of pH 7.8 (3R032), pH 7.5 (3R033) and pH 7.2 (3R034) and secondary packaging (Q=quadruplex overwrap containing aluminum; D=duplex overwrap not containing aluminum), over a 9-month period under long-term stability study conditions (25±2° C./40±5% RH).

FIG. 11 shows a graph comparing pH measurements of 12.6 mg/mL sodium bicarbonate solutions with pH targets of pH 7.8 (3R032), pH 7.5 (3R033) and pH 7.2 (3R034) and secondary packaging (Q=quadruplex overwrap containing aluminum; D=duplex overwrap not containing aluminum), over a 9-month period under long-term stability study conditions (25±2° C./40±5% RH).

FIG. 12 shows a graph comparing pH measurements of 12.6 mg/mL sodium bicarbonate solutions with pH targets of pH 7.8 (3R032), pH 7.5 (3R033) and pH 7.2 (3R034) with secondary packaging (Q=quadruplex overwrap containing aluminum; D=duplex overwrap not containing aluminum) over a 6-month period under accelerated stability study conditions (40±2° C./≤25% RH).

FIG. 13 shows a diagram of a manufacturing process for preparing sodium bicarbonate formulations at various pH targets.

FIG. 14 shows a graph of pH measurements and assay percentages over time during the manufacturing process for a batch of the sodium bicarbonate solution of the disclosure with a pH target of 7.8, wherein PRC1051.1 indicates the sampled pH at the beginning of the filling process of one infusion bag, PRC1051.2 indicating the middle of the filling process of the infusion bag and PRC1051.3 indicates the end of the filling process of the infusion bag.

FIG. 15 shows a graph of the UV-VIS spectra at wavelength 284 nm of batches of the ready-to-use, aseptically filled sodium bicarbonate product of the present disclosure.

### DETAILED DESCRIPTION

The present invention involves sodium bicarbonate prepared in a premixed, ready-to-use, formulation that does not require reconstitution or dilution prior to administration to a patient, and remains stable and active after prolonged storage. In some aspects, the sodium bicarbonate formulation is contained in an aseptically filled, sterile infusion container. Such premixed formulations avoid the cost, inconvenience, risk of contamination, and inaccurate dosage amounts that can be associated with reconstituting or diluting a hypertonic sodium bicarbonate formulation prior to administration to a patient. In some aspects, the present invention includes ready-to-use sodium bicarbonate formulations that do not comprise a preservative or antimicrobial but remain sterile and stable when stored at room temperature for a prolonged period (e.g., 6 months, 9 months, 12 months, 18 months, 24 months, or longer periods) after being aseptically filled in an infusion container. In some aspects, the present invention includes ready-to-use sodium bicarbonate formulations that do not comprise a preservative or antimicrobial but remain sterile and substantially free of 5-hydroxymethylfurfural (5-HMF) when stored at room temperature for a prolonged period (e.g., 6 months, 9 months, 12 months, 18 months, 24 months, or longer periods) after being aseptically filled in an infusion container. In some aspects, the present invention includes ready-to-use sodium bicarbonate formulations that do not comprise a preservative or antimicrobial but remain sterile and substantially free of 5-HMF when stored at room temperature for a prolonged period (e.g., 6 months, 9 months, 12 months, 18 months, 24 months, or longer periods) after being aseptically filled in an infusion container, wherein the ready-to-use sodium bicarbonate solution comprises substantially the entire volume of the container with headspace minimized. For example, the sodium bicarbonate solution may be filled into the infusion container such that headspace occupies less than 10%, less than 9%, less than 8%, less than 7%, less than 6%, less than 5%, less than 4%, less than 3%, or less than 2% of the full container volume. In some aspects, the present invention includes ready-to-use sodium bicarbonate formulations that do not comprise a preservative or antimicrobial but remain sterile and substantially free of 5-HMF when stored at room temperature for a prolonged period (e.g., 6 months, 9 months, 12 months, 18 months, 24 months, or longer periods) after being aseptically filled in an infusion container, wherein the ready-to-use sodium bicarbonate solution comprises at least 95% of the entire volume of the container with no more than 5% headspace remaining. In some aspects, the infusion container is further packaged in an overwrap and does not require vacuum sealing the overwrap over the infusion bag. In some aspects, the overwrap film comprises aluminum.

The premixed ready-to-use products and compositions of sodium bicarbonate of the present invention are formulated for administration to a patient without the need to reconstitute or dilute the composition prior to administration.

In one aspect, the present invention relates to a sodium bicarbonate product, which comprises an aqueous sodium bicarbonate solution comprising about 0.1 to 0.2 mEq/mL sodium bicarbonate, a tonicity agent, and water for injection, wherein the sodium bicarbonate solution is packaged in an infusion container and the infusion container is further packaged in an overwrap, wherein the sodium bicarbonate product is sterile and ready-to-use and has a shelf-life of at least 6 months when stored at a controlled room temperature of 25° C.±2° C. with relative humidity (RH) at 40%±5%. In some aspects, the sodium bicarbonate is sterile and ready-to-use and has a shelf-life of at least 12 months, or at least 24 months, when stored at a controlled room temperature of 25° C.±2° C. with relative humidity (RH) at 40%±5%.

In some aspects, the sodium bicarbonate solution is aseptically filled into the infusion container. In some aspects, the infusion container is an intravenous infusion bag. In some aspects, the intravenous infusion bag comprises a flexible multilayer film. In some aspects, the flexible multilayer film comprises maleic anhydride and/or poly(ethylene-co-1-octene). In some aspects, the flexible multilayer film comprises 2 to 7 layers. In some aspects, the flexible multilayer film comprises 3 to 7 layers, 3 to 6 layers, 3 to 5 layers. In some aspects, the flexible multilayer film comprises an outer, a middle, and a fluid contact layer. In some aspects, one or more layers independently comprise poly(ethylene-co-1-octene), polyethylene (PE), and/or maleic anhydride. In one aspect, the infusion bag does not contain silicon oxide (SiOₓ).

In some aspects, the flexible multilayer film comprises three layers. In some aspects, at least one layer comprises polyethylene. In some aspects, the flexible multilayer film is made of three layers of co-extruded film. In some aspects all layers of the multilayer film have a combined thickness of about 325 μm+/−10%. In some aspects, the inner film layer, which is contact with the sodium bicarbonate solution, has a thickness of about 245 μm and comprises poly(ethylene-co-1-octene). In some aspects, the middle film layer has a thickness of about 20 μm and comprises maleic anhydride grafted polyethylene. In some aspects, the outer film layer, has a thickness of about 60 μm and comprises polyethylene (PE).

In some aspects, the flexible multilayer film comprises five layers. In some aspects, at least one layer comprises polypropylene. In some aspects, the flexible multilayer film comprises cyclic olefin polymer (COP). In some aspects, the flexible multilayer film is made of five layers of co-extruded film and comprises polypropylene. In some aspects, the each film layer has a thickness of about 200 μm+/−10%. In some aspects all layers of the multilayer film have a combined thickness of about 200 μm+/−10%.

In some aspects, the sodium bicarbonate is at a concentration of 0.1 mEq/mL. In some aspects, the sodium bicarbonate is at a concentration of 0.15 mEq/mL. In some aspects, the sodium bicarbonate is at a concentration of 0.2 mEq/mL. In some aspects, the tonicity agent is dextrose (i.e., dextrose monohydrate). In some aspects, the dextrose is at a concentration of about 50 mg/mL. In some aspects, the tonicity agent is sodium chloride or potassium chloride. In some aspects, the tonicity agent is sodium chloride at a concentration about 8 mg/mL or about 9 mg/mL. In some aspects, the sodium bicarbonate solution is substantially free of preservatives, substantially free of antimicrobials, or substantially free of both preservatives and antimicrobials. In some aspects, the sodium bicarbonate solution is substantially free of antioxidants.

In some aspects, the pH of the sodium bicarbonate solution is about 7.0 to 8.5, 7.1 to 8.5, 7.2 to 8.5, 7.3 to 8.5, 7.4 to 8.5, 7.5 to 8, 7.5 to 8.1, 7.5 to 8.2, 7.5 to 8.5, or 7.8 to 8.5. In some aspects, the pH is maintained within a range of 7.0 to 8.5 following storage for 6 months at a controlled room temperature of 25° C.±2° C. with relative humidity (RH) at 40%±5%. In some aspects, the pH is maintained within a range of 7.8 to 8.5 following storage for 6 months at a controlled room temperature of 25° C.±2° C. with relative humidity (RH) at 40%±5%. In some aspects, the pH is maintained within a range of 7.5 to 8.5 following storage for 6 months at a controlled room temperature of 25° C.±2° C. with relative humidity (RH) at 40%±5%. In some aspects, the pH is maintained within a range of 7.0 to 8.5 following storage for 12 months or more at a controlled room temperature of 25° C.±2° C. with relative humidity (RH) at 40%±5%. In some aspects, the pH is maintained within a range of 7.5 to 8.5 following storage for 12 months or more at a controlled room temperature of 25° C.±2° C. with relative humidity (RH) at 40%±5%. In some aspects, the pH is maintained within a range of 7.5 to 8.2 following storage for 12 months or more at a controlled room temperature of 25° C.±2° C. with relative humidity (RH) at 40%±5%. In some aspects, the pH is maintained within a range of 7.8 to 8.5 following storage for 12 months or more at a controlled room temperature of 25° C.±2° C. with relative humidity (RH) at 40%±5%.

In some aspects, the sodium bicarbonate solution is stable for at least 24 months when stored at a controlled room temperature when stored at a controlled room temperature of 25° C.±2° C. with relative humidity (RH) at 40%±5%. In some aspects, the pH is maintained within a range of 7.0 to 8.5 following storage for 24 months at a controlled room temperature of 25° C.±2° C. with relative humidity (RH) at 40%±5%. In some aspects, the pH is maintained within a range of 7.5 to 8.5 following storage for 24 months at a controlled room temperature of 25° C.±2° C. with relative humidity (RH) at 40%±5%.

In some aspects, the infusion bag is contained in an overwrap. In some aspects the overwrap is not vacuum sealed and the product comprises air between the infusion container and the overwrap film. In some aspects, the overwrap film comprises aluminum.

In one aspect, the sodium bicarbonate product of the present disclosure comprises an aqueous sodium bicarbonate solution comprising sodium bicarbonate, a tonicity agent, and water for injection, wherein the sodium bicarbonate product is sterile and ready-to-use, and wherein the sodium bicarbonate solution is contained in an infusion bag, wherein the infusion bag comprises a flexible multilayer film wherein at least one layer comprises maleic anhydride and/or poly(ethylene-co-1-octene).

In some aspects, the concentration of sodium bicarbonate is 0.1 mEq/mL to 0.15 mEq/mL. In some aspects, the infusion bag has less than 0.2 cm3/m2·day·bar of carbon dioxide permeability. In some aspects, the flexible multilayer film comprises polyethylene and/or maleic anhydride.

In some aspects, the volume of sodium bicarbonate solution in the infusion bag is 200 mL. In some aspects, the volume of sodium bicarbonate solution in the infusion bag is 500 mL. In some aspects, the volume of sodium bicarbonate solution in the infusion bag is 1 L.

In some aspects, the sodium bicarbonate solution is aseptically filled into the infusion container. In some aspects, the sodium bicarbonate solution is aseptically filled into the infusion container in the presence of carbon dioxide.

In some aspects, the aqueous sodium bicarbonate solution is hypertonic. In some aspects, the aqueous sodium bicarbonate solution has an osmolality of about 400-550 mOsmol/kg, or an osmolality of about 400-525 mOsmol/kg. In some aspects, the osmolality is about 500-580 mOsmol/kg. In some aspects, the amount of bacterial endotoxin in the aqueous sodium bicarbonate solution is 25 EU/mg or less.

In one aspect, the primary packaging for the sodium bicarbonate product of the present disclosure is an intravenous bag. In one aspect, the primary packaging for the sodium bicarbonate product is a pre-filled syringe. In one aspect, the primary packaging for the sodium bicarbonate product is a plastic bottle or semi-flexible IV container. In one aspect, the primary packaging for the sodium bicarbonate product of the present disclosure is a glass container.

In one aspect, the intravenous bag is made up of multilayer polymer film that is compatible with the dilute sodium bicarbonate solution of the present disclosure. Through the experiments disclosed herein, the inventors have determined that containers available commercially under the Renolit IV bag products manufactured by Renolit, such as Renolit Solmed Infuflex 9101 and Renolit MF518H possess desirable properties and are compatible with the dilute sodium bicarbonate solution of the present disclosure.

In some aspects, the infusion bag of the sodium bicarbonate product of the present disclosure comprises at least one port. In some aspects, the infusion bag of the sodium bicarbonate product of the present disclosure comprises at least two ports.

In some aspects, the infusion bag of the sodium bicarbonate product of the present disclosure comprises at least one closure. In certain aspects the closure is a twist-off closure. In certain aspects, the closure comprises a membrane that creates a barrier, splitting the closure in two parts. In certain aspects, the closure comprises an inferior part of the membrane in direct contact with the sodium bicarbonate solution, while the superior part is in contact with a zone that forms an air chamber into the closure. In some aspects, the twist-off closure comprises PE, polypropylene PP or a combination thereof. In certain aspects, other polymers that are stable, with low leachables, and without physical deformation during exposure to thermal sealing may also be used for the closure. In some aspects, the closure is a rubber plug. In some aspects, the rubber plug provides a fluid tight closure of the passage and a lid member that clamps the periphery of the rubber plug. In one aspect, the rubber plug is a resealable plug which provides fluid tight closure of the passage and a plastic cap that clamps the periphery of the resealable plug. In certain aspects, the closure comprise a plastic material. In certain aspects, the closure is a cap. Other closure systems that are stable with low leachables are also contemplated in this disclosure.

In some aspects, the infusion bag of the sodium bicarbonate product of the present disclosure comprises at least one port that is a tubing port. In some aspects, the tubing port is used to fill the bag with the aqueous sodium bicarbonate solution. In certain aspects, the tubing port comprises one or more layers of a plastic material. In some aspects, the tubing port comprises a ethylene based octene-1 elastomer material. In some aspects, the tubing port comprises a ethylene based octene-1 elastomer one-layer co-extrudate material. In some aspects, the tubing port comprises a single-layer co-extruded connector tubing. In some aspects, the tubing port is PVC and/or plasticizer free. In some aspects, the tubing port is sealed closed.

In one aspect, the infusion bag comprises a flexible film. In some aspects, the flexible film may be a multilayer film or a single layer film. In some aspects, the infusion bag of the sodium bicarbonate product of the present disclosure comprises a flexible multilayer PE film. In some aspects the infusion bag comprises 2 to 7 layers of PE film. In some aspects, the infusion bag comprises a 3 layer PE film. In some aspects, the flexible multilayer film comprises PE and maleic anhydride grafted PE layers. In some aspects, the flexible multilayer film comprises PE and poly(ethylene-co-1-octene) layers. In some aspects, the flexible multilayer film comprises PE, maleic anhydride grafted PE, and poly(ethylene-co-1-octene) layers. In some aspects, the innermost layer of the infusion container is made-up of a material that does not adsorb sodium bicarbonate thereby causing no loss of potency and/or assay percentage during preparation, sterilization, and storage. In some aspects, the innermost layer of the infusion container, which is in contact with the sodium bicarbonate solution, comprises poly(ethylene-co-1-octene). In some aspects, one or more layers of the multilayer film comprise maleic anhydride. In some aspects, the layer comprising maleic anhydride is not in contact with the sodium bicarbonate solution. In some aspects, at least one layer of the multilayer film is made up of a material that is impermeable to carbon dioxide, thereby maintaining a consistent pH in the sodium bicarbonate solution during storage and administration. In some aspects, the multilayer film does not comprise SiOₓ. In some aspects, the multilayer film is a Renolit Solmed Infuflex film. In some aspects, the multilayer film is a Renolit MF518H film. In some aspects, the multilayer film is a Renolit Solmed Infuflex 9101 film, made up of an inner layer of poly(ethylene-co-1-octene), a middle layer of maleic anhydride grafted PE and an outer layer of PE. In some aspects, the multilayer film is a Renolit MF518H film comprising polypropylene. In certain aspects, other polymers that are stable, with low leachables, impermeable to carbon dioxide and without physical deformation during sterilization may also be used for the infusion bag. In certain aspects, the infusion bag comprising a flexible film has a water vapor transmission rate between 1.3-1.7 g/(m2·24 h). In certain aspects, the infusion bag comprising a flexible film has an oxygen permeability between 380-400 cm3·(m2·24 h·0.1 MPa). In certain aspects, the infusion bag comprising a flexible film has nitrogen permeability between 78-82 cm3·(m2·24 h·0.1 MPa). In certain aspects, the infusion bag comprising a flexible film has a tensile strength of 20(MD)/20(TD) MPa. In certain aspects, the infusion bag comprising a flexible film has transmittance between 88-92%. In some aspects, the infusion bag further comprises additives. In some aspects, the additives are a drying agent, antioxidant, or a stabilizer.

In a preferred embodiment, the infusion container comprising the flexible multilayer film comprises poly(ethylene-1-co-octene) and/or maleic anhydride grafted PE. In some embodiments, the infusion container comprising the flexible multilayer film comprises polypropylene. In some embodiments, the infusion container comprising the flexible multilayer film comprises ethylene vinyl alcohol (EVOH).

In some aspects, the multilayer film may comprise layers made up of materials such as polyethylene or polypropylene. In some aspects, the inner layer of the multilayer film comprises poly(ethylene-co-1-octene). In some aspects, the multilayer film may comprise one or more middle film layers comprising maleic anhydride grafted polyethylene (PE). In some aspects, the outer layer of the multilayer film comprises PE. In some aspects, the flexible multilayer film comprises five layers each comprising polypropylene. In some aspects, the flexible multilayer film comprises polyethylene and EVOH.

In some aspects, the infusion bag further comprises an overwrap. In some aspects, the overwrap is a multilayer overwrap comprising two, three, four, or five layers. In some aspects, the overwrap is a duplex overwrap. In some aspects, the overwrap is a quadruplex wrap. In some aspects, the overwrap comprises aluminum. In some aspects, the overwrap comprises polyester, aluminum, polypropylene, or a combination thereof. In some aspects, the overwrap is vacuum sealed over the primary packaging. In some aspects, the overwrap is not vacuum sealed over the primary packaging. For example, the solution of the sodium bicarbonate is disposed in an infusion bag, which is packaged in a secondary packaging, which is not vacuum sealed over the secondary packaging.

In one aspect, the sodium bicarbonate is chemically stable for at least 12 months when stored at a controlled room temperature. In one aspect, the controlled room temperature is 25° C.±2° C. In one aspect, the controlled room temperature is 25° C.±2° C. with relative humidity (RH) at 40%±5%.

In one aspect, the sodium bicarbonate solution is free of visible particles when the sodium bicarbonate product of the present disclosure comprising an aqueous sodium bicarbonate solution is at room temperature for at least 12 months. In certain aspects, the sodium bicarbonate solution is free of visible particles when the sodium bicarbonate product of the present disclosure comprising an aqueous sodium bicarbonate solution is at room temperature for at least 9 months. In certain aspects, the sodium bicarbonate solution is free of visible particles when the sodium bicarbonate product of the present disclosure comprising an aqueous sodium bicarbonate solution is at room temperature for at least 6 months.

In one aspect, the bicarbonate content after long term stability at 25° C.±2° C. with relative humidity (RH) at 40%±5% for 6 months is between 95-105% of the initial value. In one aspect, the bicarbonate content after long term stability at 25° C.±2° C. with relative humidity (RH) at 40%±5% for 6 months is between 90-110% of the initial value. In one aspect, the bicarbonate content after long term stability at 25° C.±2° C. with relative humidity (RH) at 40%±5%. for 3 months is between 95-105% of the initial value. In one aspect, the bicarbonate content after long term stability at 25° C.±2° C. with relative humidity (RH) at 40%±5% for 3 months is between 90-110% of the initial value. In one aspect, the bicarbonate content after long term stability at 25° C.±2° C. with relative humidity (RH) at 40%±5% for 9 months is between 95-105% of the initial value. In one aspect, the bicarbonate content after long term stability at 25° C.±2° C. with relative humidity (RH) at 40%±5% for 9 months is between 90-110% of the initial value. In one aspect, the bicarbonate content after long term stability at 25° C.±2° C. with relative humidity (RH) at 40%±5%. for 24 months is between 95-105% of the initial value. In one aspect, the bicarbonate content after long term stability at 25° C.±2° C. with relative humidity (RH) at 40%±5% for 9 months is between 90-110% of the initial value.

In one aspect, the infusion bag is aseptically filled with the sodium bicarbonate solution.

In one aspect, the present invention relates to a method for treating metabolic acidosis in a patient in need thereof, comprising administering the aqueous sodium bicarbonate product of the present disclosure to the patient over a time period of about 4 to 8 hours, wherein the pH of the sodium bicarbonate solution changes less than 0.2 or 0.1 pH units during the infusion time.

In one aspect, the present invention relates to a method for treating metabolic acidosis in a patient in need thereof, comprising administering the aqueous sodium bicarbonate product of the present invention to the patient over a time period of about 12 to 24 hours, wherein the pH of the sodium bicarbonate solution changes less than 0.1 pH units during the infusion time.

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the present disclosure and how to make and use them.

As used herein, the term “antioxidant” refers to a compound that can reduce oxidative processes and thereby stabilize pharmaceutical formulations. Non-limiting examples of antioxidants include histamine, methionine, ascorbic acid, sodium ascorbate, glutathione, vitamin E, vitamin C, poly(ethylenimine), N-acetyl cysteine, S-adenosylmethionine, sodium metabisulfite, propyl gallate, alpha-tocopherol, bisulfite or a combination thereof. As used herein, the term “antioxidant” does not include dextrose.

As used herein, the term “significantly free of,” with respect to a compound or category of compounds, means that compound or category of compounds is not added to the sodium bicarbonate solution of the present invention.

The term, “chemically stable,” as used herein, refers to a chemical compound which retains its chemical structure and useful properties on a timescale of its expected usefulness. Specifically, the usefulness of the compound is maintained in the environment in which it is stored. Conversely, a compound lacks chemical stability if it decomposes under the conditions of a specific environment. As used herein, in certain embodiments, “chemically stable” may mean resistant to degradation of sodium bicarbonate into its known or unknown decomposition elements.

As used herein, the terms “premix”, “premixed”, or “premixture” refers to a pharmaceutical formulation that does not require reconstitution or dilution prior to administration to a patient. For example, in contrast to non-premixed formulations of sodium bicarbonate, the premixed compositions provided herein are suitable for administration to a patient without dilution by, for example, a clinician, hospital personnel, caretaker, patient or other individual.

As used herein, the term “ready-to-use” refers to premixed compositions that are suitable for administration to a patient without further manipulation (e.g., a pharmaceutical formulation that is in the container from which the product is administered to the patient (such as an infusion bag or prefilled syringe) and does not require dilution or admixing before administration).

As used herein, the term “container closure system” refers to the sum of packaging components that together contain and protect the dosage form, including primary packaging components and secondary packaging components.

As used herein, the terms “primary packaging components” and “primary packaging” mean packaging components that are or may be in direct contact with the dosage form. For example, the primary packaging may be an infusion bag, including a flexible film component, one or more ports, and a closure system.

The terms “infusion bag,” “intravenous bag,” and “IV bag” may be used interchangeably herein and refer to a type of primary packaging, which comprises a bag film and optionally port(s) and closure system(s). Such infusion bags may be used to administer intravenous infusion products.

The term “infusion container” as used herein refers to a type of primary packaging used to administer intravenous infusion products, for example, an infusion bag, a semi-flexible plastic infusion container, a prefilled syringe, a glass infusion container, etc.

As used herein, the term “flexible” refers to a material that has the ability to deform and return to its original shape when the applied stress is removed without breaking or damage at around standard temperature and pressure (STP), for example about 0° C.±40° C. and 1 atm±0.005 atm. Flexible materials are distinguished from rigid materials such as glass. A “flexible” infusion bag allows fluid to drain out of the bag without venting. A “flexible” infusion bag may have port tubes and/or closures that are made of rigid or semi-rigid materials, provided that the bag film is flexible.

As used herein, the terms “secondary packaging components” and “overwrap” refer to a packaging component that is intended to provide additional protection to the drug product but is not typically in direct contact with the dosage form during storage.

As used herein, the terms “subject” or “patient” refer to a human, a non-human mammal or a non-human animal. The compounds and compositions of the present disclosure have application in veterinary medicine as well as for humans, e.g., for the treatment of domesticated species such as canine, feline, and various other pets; farm animal species such as bovine, equine, ovine, caprine, porcine, etc.; wild animals, e.g., in the wild or in a zoological garden; and avian species, such as chickens, turkeys, quail, songbirds, etc.

As used herein, the term “shelf life” refers to the time period during which a pharmaceutical formulation is expected to remain stable, or retain its identity, strength, quality, and purity, when it is properly stored according to storage conditions supported by stability testing.

As used herein, the terms “pharmaceutical composition” or “pharmaceutical formulation” relates to compositions that are formulated using one or more pharmaceutically acceptable diluents or excipients. A “pharmaceutically acceptable” solvent, diluent or excipient, as used herein, means approved by a regulatory agency of a federal or a state government, or as listed in the U.S. Pharmacopoeia or other generally recognized pharmacopoeia for use in mammals, and more particularly in humans.

As used herein, the term “titrated to effect” refers to a method of limiting potential side effects of a drug by administering the smallest dosage needed to obtain the desired effects in a patient, which can be readily determined according to standard good medical practice by those of skill in the art. In a non-limiting embodiment, a medication is titrated to effect by starting a medication at a low dose and slowly increasing the dose until the maximum effective dose is achieved or until side effects occur.

As used herein, the terms “about” and “approximately” as used herein means within an acceptable error range for the particular value as determined by one of ordinary skill in the art, which will depend in part on how the value is measured or determined, i.e., the limitations of the measurement system. In certain aspects, “about” can mean a range of up to 10% of a given value.

As used herein, the terms “aseptically filled” and “aseptic processing” refers to sterile filtration and aseptic processing of the premixed sodium bicarbonate solution of the disclosure, wherein the pre-filtration followed by sterile filtration is used for sterilization of solution. For example, aseptic filling can be achieved by filtering the premixed sodium bicarbonate solution of the disclosure through a pre-filter with a filter absolute rating: 0.45 μm followed by a filtration through two sterilizing filters (0.2 μm pore size filters) under aseptic conditions. The filtration can be pressure driven (conducted under compressed air) and the product solution can be aseptically filled into presterilized bag in line with filtration. After filling, port of the IV bags used for product solution filling can be sealed, e.g., thermally sealed.

As used herein, the terms “port,” “tubing port,” “port tube,” and “connector tubing” refer to a connector which is sealed into the container portion of an infusion bag. An infusion bag may include one or more ports. The ports may include administrative and/or additive ports. The ports of the invention use commercially available polymers, elastomers, etc. and can comprise one or more layers. The ports may be coextruded.

As used herein, the term “closure” refers to a component that closes or seals a container. A closure provides an effective barrier against microbial contamination. In some aspects, the closure is a twist-off closure. The closures referred to herein may comprise commercially available polymers, elastomers, etc. and can comprise one or more layers.

As used herein, singular forms, including the singular forms “a” “an” and “the”, specifically also encompass the plural referents of the terms to which they refer unless the context clearly dictates otherwise. In addition, as used herein, unless specifically indicated otherwise, the word “or” is used in the “inclusive” sense of “and/or” and not the “exclusive” sense of “either/or”.

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps but may include additional steps. When used in the context of a compound or composition, the term “comprising” means that the compound or composition includes at least the recited features or components but may also include additional features or components.

### Pharmaceutical Formulations

The compounds and compositions of the invention may be formulated as pharmaceutical compositions by admixture with pharmaceutically acceptable solvents and excipients. For example, suitable components of pharmaceutical compositions are described in, for example, “Remington's Pharmaceutical Sciences” by Philip P. Gerbino, 21st Edition (or previous editions). In certain non-limiting embodiments, the compounds or compositions are provided in a therapeutically effective amount to an animal, such as a mammal, preferably a human, in need of treatment therewith, e.g., for treatment of metabolic acidosis, drug overdose, or severe diarrhea.

In certain non-limiting embodiments, sodium bicarbonate is formulated as a pharmaceutical composition, wherein the sodium bicarbonate is the only active pharmaceutical ingredient present in the composition. In another non-limiting embodiments, sodium bicarbonate is formulated as a pharmaceutical composition, wherein the sodium bicarbonate is formulated in combination with at least one or more other biologically active ingredients. The formulation is suitable for parenteral administration, such as intravenous, subcutaneous, intramuscular and intraperitoneal administration.

The pharmaceutical formulations suitable for injectable use, such as, for example, intravenous administration, include sterile aqueous solutions or dispersions and sterile powders for the extemporaneous preparation of sterile injectable solutions or dispersion. The pharmaceutical formulations can be stable under the conditions of manufacture and storage and can be preserved against the contaminating action of microorganisms such as bacteria and fungi. Solvents or dispersion mediums suitable for stable pharmaceutical formulations include, for example, water for injection (WFI), saline, aqueous dextrose, ethanol, polyol (for example, glycerol, propylene glycol, and polyethylene glycol, and the like), suitable mixtures thereof, and oils. In certain aspects, the solvent may be sterile WFI, 0.9% sodium chloride injection (normal saline), or 5% dextrose in water.

Pharmaceutical formulations may include tonicity adjusting agents, for example, sugars or water-soluble inorganic salts including but not limited to dextrose, glycerin, glycerol, mannitol, trehalose, sorbitol, lactose, trehalose, potassium chloride, sodium chloride, sodium sulfate, and combinations thereof. Sterile injectable solutions may be prepared by incorporating the sodium bicarbonate in the required amounts in the appropriate solvent with various of the other ingredients enumerated above, as required, followed by filter sterilization.

Pharmaceutical formulations may include one or more excipients. Pharmaceutically acceptable excipients which may be included in the formulation are tonicity agents such as dextrose, glycerin, mannitol, trehalose, sodium chloride, and potassium chloride; added buffers such as Tris, HEPES, Tricine, Gly-Gly; amino acids; urea; alcohols; ascorbic acid; phospholipids; proteins, such as serum albumin, collagen, and gelatin; chelating agents such as EDTA or EGTA; liposomes; polyvinylpyrollidone; dextranpropylene glycol and polyethylene glycol (e.g., PEG-4000, PEG-6000); glycerol; glycine; lipids; preservatives; antimicrobials; suspending agents; stabilizers; and dyes. The term “stabilizer” refers to a compound optionally used in the pharmaceutical compositions of the present invention to increase storage life.

In some aspects, formulations of the present disclosure are substantially free of antioxidants. Non-limiting examples of antioxidants include histamine, methionine, ascorbic acid, sodium ascorbate, glutathione, vitamin E, vitamin C, poly(ethylenimine), N-acetyl cysteine, S-adenosylmethionine, sodium metabisulfite, propyl gallate, alpha-tocopherol, bisulfite or a combination thereof. As used herein, the term “antioxidant” does not include dextrose. Thus, in some aspects, formulations of the present disclosure do not contain one or more antioxidants.

Pharmaceutical formulations may include one or more non-ionic surfactants. Examples of non-ionic surfactants include polysorbate 20, polysorbate 80, Triton™ X-100 (t-Octylphenoxypolyethoxyethanol, Polyethylene glycol tert-octylphenyl ether), Triton™ X-114 ((1,1,3,3-Tetramethylbutyl)phenyl-polyethylene glycol, Polyethylene glycol tert-octylphenyl ether), Nonidet™ P-40 (4-Nonylphenyl-polyethylene glycol, Imbentin-N/52, NP 40), Octyl α-glucoside, Octyl β-glucoside, Brij® 35 (Polyoxyethylene lauryl ether), Pluronic™ (Non-ionic copolymer surfactant).

Pharmaceutical formulations of the present disclosure may have a buffer capacity (β)>0.017. In some aspects, the pharmaceutical formulations may have a buffer capacity of β>0.018. In some aspects, the pharmaceutical formulations may have a buffer capacity of β>0.019. In some aspects, the pharmaceutical formulations may have a buffer capacity of β>0.02. Buffering capacity is calculated using the formula: β=n/ΔpH, wherein β is buffer capacity, n is the number of moles of a strong acid or strong base added per liter of the sodium bicarbonate solution, ΔpH is the change in pH after the addition of the strong acid or strong base to the pharmaceutical formulation. In one aspect, the buffering capacity is assessed by adding hydrochloric acid (HCl) to a sodium bicarbonate solution until pH 7.4 is reached, and then calculating the buffer capacity (β) by dividing i) the number of moles of HCl required to reach pH 7.4 by ii) the difference between the starting solution pH and pH 7.4.

### Methods of Using Ready-to-Use Sodium Bicarbonate Compositions

As noted above, exemplary methods of treatment according to the disclosure include treatment of clinically important metabolic acidosis in a patient in need thereof comprising administration of sodium bicarbonate to the patient, wherein the sodium bicarbonate is administered in an amount effective to produce the desired effect. In certain non-limiting embodiments, the dosage of sodium bicarbonate can be individualized and titrated to the desired clinical effect.

The sodium bicarbonate for use in the invention can be administered via any suitable infusion rate without the need to reconstitute or dilute the composition prior to administration. Non-limiting examples of suitable infusion rates useful in the methods of treatment according to the disclosure are about 250 ml/hr, about 125 ml/hr, 190 ml/hr to 200 ml/hr, 48 ml to 52 ml/hr, 23 ml/hr to 27 ml/hr, 15 ml/hr to 18 ml/hr, 3 ml/min, 4 ml/min, 50 ml/hr, 16 ml/hr, 17 ml/hr, 8 ml/hr and 9 ml/hr. In some aspects, the aqueous sodium bicarbonate solution of the disclosure is administered at a rate of 8 ml/hr to 250 ml/hr. In some aspects, the patient is administered less than 1.5 mmol of sodium bicarbonate per kg body weight per hour for human adults. In some aspects, the patient is administered less than 5 mmol per kg body weight per day for human pediatric patients.

### Examples

The following examples are merely illustrative of the presently disclosed subject matter and they should not be considered as limiting the scope of the present disclosure in any way.

### Example 1: Exemplary Formulations

In certain non-limiting examples, sodium bicarbonate formulations were made according to the compositions of Table 1. For 100 mEq/L solutions of sodium bicarbonate (NaHCO3), the formulation ingredients were 8.4 mg/mL sodium bicarbonate, 50 mg/mL dextrose, water for injection (q.s.), and carbon dioxide (q.s. to pH). For 150 mEq/L solutions of NaHCO3, the formulation ingredients were 12.6 mg/mL sodium bicarbonate, 50 mg/mL dextrose, water for injection (q.s.), and carbon dioxide (q.s. to pH).

TABLE 1
Sodium Bicarbonate Formulations

| | (100 mEq/L of | (150 mEq/L of |
|---|---|---|
| | NaHCO3) | NaHCO3) |
| Component | Quantity (mg/mL) | Quantity (mg/mL) |
| Sodium Bicarbonate | 8.4 | 12.6 |
| Dextrose | 50 | 50 |
| Water for injection (WFI) | q.s. | q.s |
| carbon dioxide | Optional pH | Optional pH |
| | Adjuster | Adjuster |

The present disclosure includes sodium bicarbonate products provided in various volumes with the same proportions of components as listed in Table 1 above. Further, the present disclosure includes other solvent systems that may be substituted for the 5% dextrose in water for injection (WFI). For example, approximately 9 mg/mL sodium chloride (0.9% NaCl) in water for injection may be substituted for the 5% dextrose in WFI in the above formulations. The sodium bicarbonate formulations of Table 1 are used throughout the Examples below (hereinafter “sodium bicarbonate solution of the disclosure”) and beneficially do not require a preservative or antimicrobial.

The target pH is reached by adjusting the amount of carbon dioxide added to the sodium bicarbonate solution of the disclosure. The 12.6 mg/mL sodium bicarbonate solution of Table 1 was produced in batches with different target pH levels: 3R032 (pH 7.8), 3R033 (pH 7.5) and 3R034 (pH 7.2). The buffer capacity (B) of the solutions varied depending on the pH of the formulation. The inventors have determined that the higher buffer capacity is desirable for stability of the dilute sodium bicarbonate solutions of the present disclosure.

TABLE 2
Buffering Capacity (β)

| Batch | Initial formulation pH | β |
|---|---|---|
| 3R032 | 7.8 | 0.019043 |
| 3R033 | 7.5 | 0.012 |
| 3R034 | 7.2 | 0.01 |

### Example 2: Process for Compounding of Bulk Solution

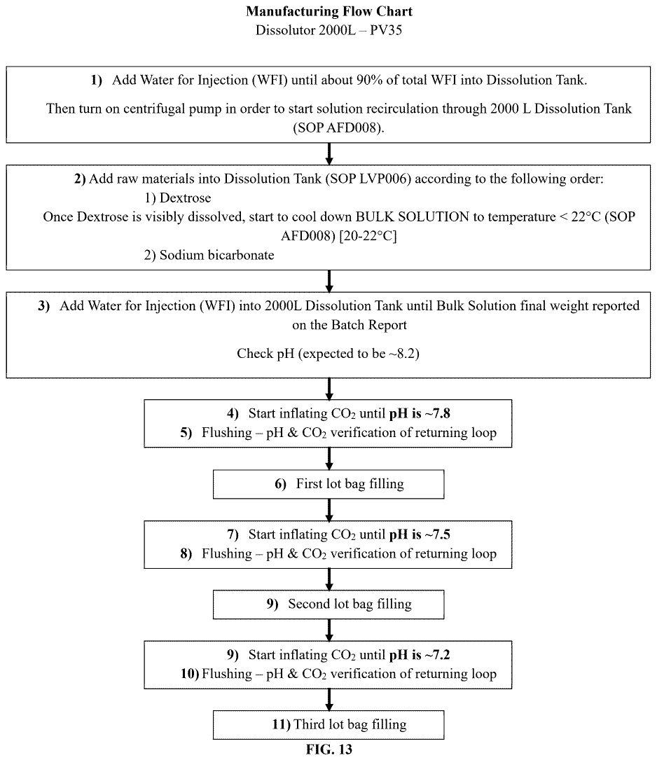

The 12.6 mg/mL sodium bicarbonate solution of Table 1 was produced at varying target pH levels. The manufacturing process is shown in FIG. 13 and involved compounding a bulk solution. For compounding of bulk solution, predefined volume (90%) of Water for Injection (WFI) was collected in the compounding vessel prior start of the compounding. The temperature of WFI and temperature of solution throughout the compounding process was maintained in the range of ≤22° C. First, the dispensed quantity of Dextrose was added. Once Dextrose was visibly dissolved, the bulk solution cooled to a target temperature of about 20° C. to 22° C. When the target temperature was reached, the dispensed quantity of Sodium Bicarbonate was added. Using carbon dioxide, the pH was adjusted to about 7.8 prior to filling Lot 3R032, about 7.5 prior to filling Lot 3R033, and about 7.2 prior to filling Lot 3R034.

After batch weight, solution was recirculated at a speed of no less than 1.7 L/s and the samples were taken from the compounding vessel after 5, 10, and 15 minutes of recirculation and analyzed for appearance, bicarbonate assay, pH, osmolality as shown in Table 3 below for three batches of the aqueous sodium bicarbonate solution of the disclosure the result of the analysis is shown in Table 4 below.

TABLE 3
Acceptance Criteria

| Test | Specifications | Units |
|---|---|---|
| Appearance | Clear and colourless solution. | — |
| | Free from visible particles | |
| Bicarbonate Assay | 95-105 | % |
| pH | 7.0-8.5 | pH Units |
| Osmolality | For information only | mOsmol/kg |
| Conductivity | For information only | mS/cm |
| Bulk Solution Density | For information only | g/ml |

TABLE 4
Dissolution and flushing results

| | | | | |
|---|---|---|---|---|
| | | | Dissolu- | Flush- |
| Test | Acceptance criteria | Units | tion | ing |

Lot 3R032 (target pH = 7.8)

| | | | | |
|---|---|---|---|---|
| Clarity and | Clear and colourless | — | Comply | Comply |
| Color | solution | | | |
| Appearance | Free from visible | — | Comply | Comply |
| | particles | | | |
| Bicarbonate | 95-105 | % | 99.56 | 97.83 |
| Assay | | | | |
| pH | 7.0-8.5 | pH Units | 7.849 | 7.871 |
| Osmolality | For information only | mOsmol/ | 536 | 534 |
| | | kg | | |
| Conductivity | For information only | mS/cm | 9.681 | 9.555 |
| Density | For information only | g/cm3 | 1.02277 | 1.02215 |

Lot 3R033 (target pH = 7.5)

| | | | | |
|---|---|---|---|---|
| Clarity and | Clear and colourless | — | Comply | Comply |
| Color | solution | | | |
| Appearance | Free from visible | — | Comply | Comply |
| | particles | | | |
| Bicarbonate | 95-105 | % | 97.71 | 97.66 |
| Assay | | | | |
| pH | 7.0-8.5 | pH Units | 7.459 | 7.426 |
| Osmolality | For information only | mOsmol/ | 533 | 532 |
| | | kg | | |
| Conductivity | For information only | mS/cm | 9.562 | 9.581 |
| Density | For information only | g/cm3 | 1.02256 | 1.02246 |

Lot 3R034 (target pH = 7.2)

| | | | | |
|---|---|---|---|---|
| Clarity and | Clear and colourless | — | Comply | Comply |
| Color | solution | | | |
| Appearance | Free from visible | — | Comply | Comply |
| | particles | | | |
| Bicarbonate | 95-105 | % | 97.97 | 97.87 |
| Assay | | | | |
| pH | 7.0-8.5 | pH Units | 7.309 | 7.287 |
| Osmolality | For information only | mOsmol/ | 539 | 543 |
| | | kg | | |
| Conductivity | For information only | mS/cm | 9.555 | 9.560 |
| Density | For information only | g/cm3 | 1.02252 | 1.02250 |

The “dissolution” data were collected 10 minutes from reaching pH target. The “flushing” data were collected after 60 kg flushing. The results confirmed that all raw materials were completely dissolved after 10 minutes and that the produced solution was well mixed by recirculation. Additionally, the solution shows no variation of Bicarbonate Assay, density, conductivity and osmolality noted at different pH targets. Additionally, the results show that the pH of the solution remained stable between dissolution and flushing.

Furthermore, finished product analysis was performed in order to evaluate the pH behavior during filling and infusion. The bicarbonate solutions prepared above were filled into flexible infusion bags (Renolit 9101). The acceptance criteria are listed in Table 5 and the results of three batches are listed in Tables 6, 7 and 8 below:

TABLE 5
Finished Product Analysis Acceptance Criteria

| Test | Specifications | Units |
|---|---|---|
| Appearance | Clear and colorless solution. | — |
| | Free from visible particles | |
| Subvisible | Particles ≥ 10 μm | particles/ml |
| particles* | →25/ml Particles ≥ 25 | |
| | μm →3/ml | |
| Bicarbonate Assay | 95-105 | % |
| pH (start and End of | 7.0-8.5 | pH Units |
| filling) | | |
| Osmolality | For information only | mOsmol/kg |
| Conductivity | For information only | mS/cm |

TABLE 6
Lot 3R032 Finished Product Analysis Results

| | | Lot 3R032 (target pH = 7, 8) | | | | |
|---|---|---|---|---|---|---|
| | | Flushing | Filling | | | |
| Test | Acceptance criteria | (t = 0) | (beginning) | (middle) | (end) | Δ% |
| Clarity and Color | Clear and | Comply | | | | |
| | colourless | | | | | |
| | solution | | | | | |
| Appearance | Free from visible | Comply | | | | |
| | particles | | | | | |
| Bicarbonate | 95-105% | 97.83 | 97.72 | 97.78 | 97.87 | 0.08 |
| Assay | | | | | | |
| pH | 7.0-8.5 | 7.871 | 7.907 | 7.882 | 7.827 | 0.57 |
| Osmolality | [mOsmol/kg] | 534 | 525 | 524 | 529 | 1.14 |
| Conductivity | [mS/cm] | 9.555 | 9.571 | 9.576 | 9576 | 0.15 |

TABLE 7
Lot 3R033 Finished Product Analysis Results

| | | Lot 3R033 (target pH = 7.5) | | | | |
|---|---|---|---|---|---|---|
| | Acceptance | Flushing | Filling | | | |
| Test | criteria | (t = 0) | (beginning) | (middle) | (end) | Δ% |
| Clarity and Color | Clear and | Comply | | | | |
| | colourless | | | | | |
| | solution | | | | | |
| Appearance | Free from visible | Comply | | | | |
| | particles | | | | | |
| Bicarbonate | 95-105% | 97.66 | 97.78 | 98.16 | 97.97 | 0.27 |
| Assay | | | | | | |
| pH | 7.0-8.5 | 7.426 | 7.461 | 7.454 | 7.448 | 0.29 |
| Osmolality | [mOsmol/kg] | 532 | 533 | 534 | 534 | 0.23 |
| Conductivity | [mS/cm] | 9.581 | 9.568 | 9.565 | 9.560 | 0.13 |

TABLE 8
Lot 3R034 Finished Product Analysis Results

| | | Lot 3R034 (target pH = 7.2) | | | | |
|---|---|---|---|---|---|---|
| | Acceptance | Flushing | Filling | | | |
| Test | criteria | (t = 0) | (beginning) | (middle) | (end) | Δ% |
| Clarity and Color | Clear and | Comply | | | | |
| | colourless | | | | | |
| | solution | | | | | |
| Appearance | Free from visible | Comply | | | | |
| | particles | | | | | |
| Bicarbonate | 95-105% | 97.87 | 98.02 | 97.92 | 97.88 | 0.10 |
| Assay | | | | | | |
| pH | 7.0-8.5 | 7.287 | 7.317 | 7.326 | 7.334 | 0.40 |
| Osmolality | [mOsmol/kg] | 543 | 538 | 537 | 539 | 0.70 |
| Conductivity | [mS/cm] | 9.560 | 9.553 | 9.556 | 9.569 | 0.10 |

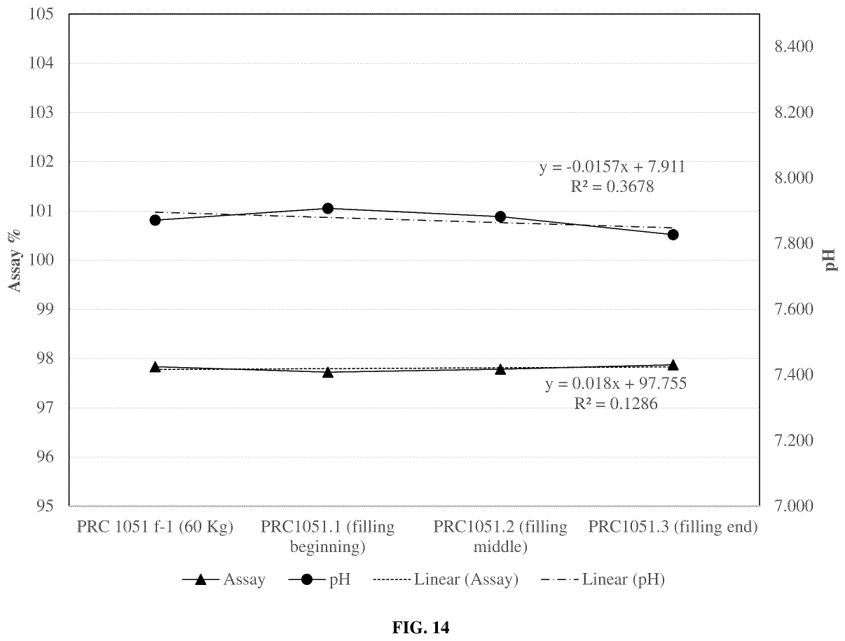

The pH of the solution was measured during the filling process and is shown in FIG. 14. The results show that the finished product (hereinafter “the ready-to-use, aseptically filled sodium bicarbonate product of the present disclosure”) remained stable during the filling process.

### Example 3: Container pH Study

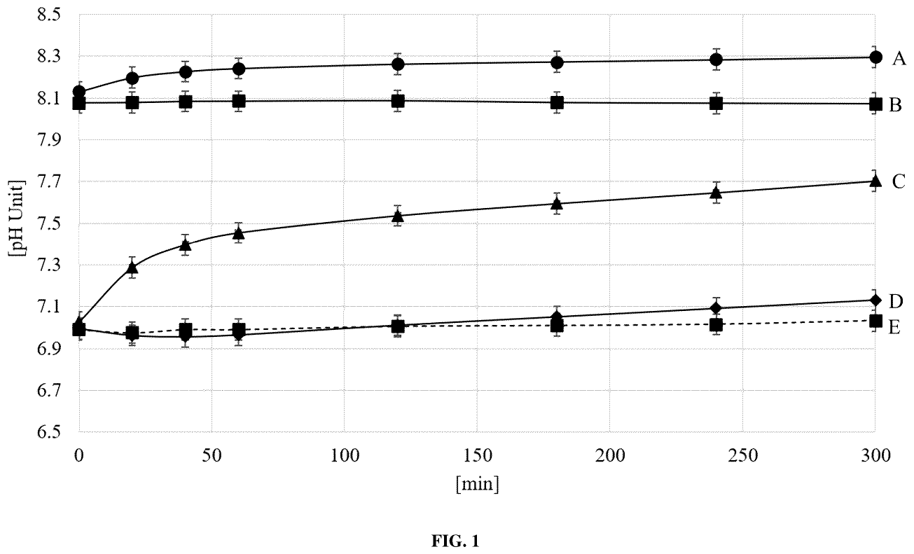

The pH stability of the 12.6 mg/mL sodium bicarbonate solution of Table 1 was evaluated using a glass vial as the primary packaging, with and without an initial pH adjustment using carbon dioxide and carbon dioxide headspace (HS). The results show that the pH is more stable in the batches without headspace and when the headspace is filled with carbon dioxide. The pH increase is more significant when the pH is modified with carbon dioxide to approximately 7. The results are shown in FIG. 1.

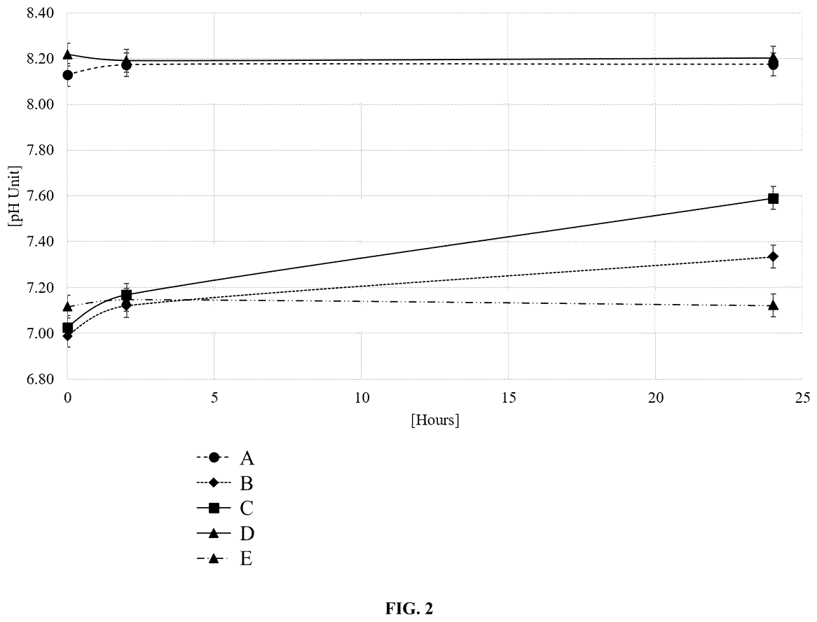

Furthermore, the pH of the 12.6 mg/mL sodium bicarbonate solution of Table 1 was evaluated using three different commercially available infusion bags (Renolit Solmed Infuflex 9101, Renolit MF518H, and Nexcel M312A), with and without an initial pH adjustment to pH 7.0 using carbon dioxide. The results show that the pH increase is less significant if the initial pH is not adjusted to 7.0 with carbon dioxide. The results are shown in FIG. 2. As shown in FIG. 2, the 12.6 mg/mL sodium bicarbonate solution of Table 1 contained in the Nexcel bag showed significant change in pH while the 12.6 mg/mL sodium bicarbonate solution of Table 1 did not show significant changes in pH when stored in the Renolit Solmed Infuflex 9101 and Renolit MF518H bags.

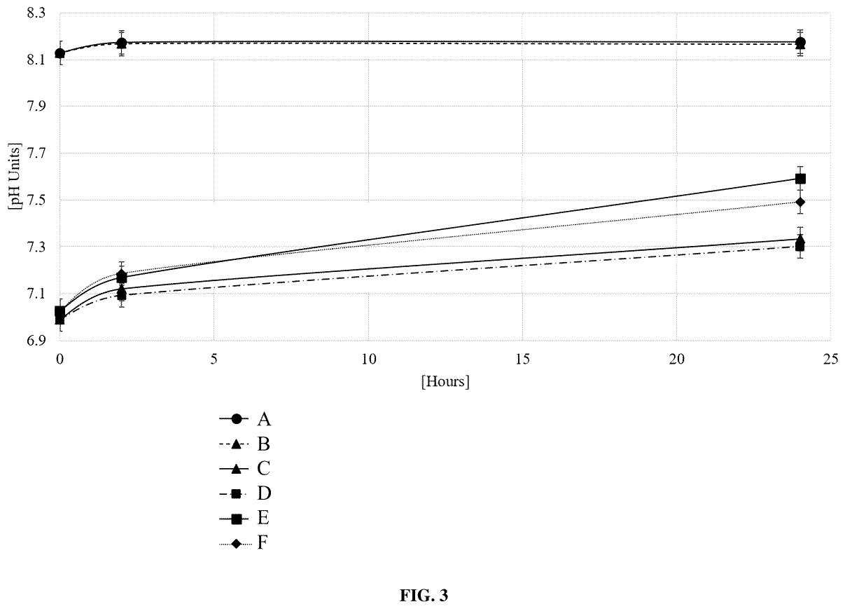

The pH of the ready-to-use 12.6 mg/mL sodium bicarbonate solution of Table 1 contained in Renolit MF518H and Nexcel M312A infusion bags with and without an overwrap (OW) and with and without an initial pH adjustment was evaluated over a 24 hour period. The results are shown in FIG. 3. The results show that there are not significant differences with and without overwrap for Renolit MF518H during this 24 hour study. For Nexcel M312A, the pH of the solution is more stable with overwrap. Similarly, the 12.6 mg/mL sodium bicarbonate solution of Table 1 contained in the Nexcel bag with and without an overwrap and with pH adjustment to pH 7.0 showed the significant change in pH, with the Nexcel bag without an overwrap showing the most significant change in pH.

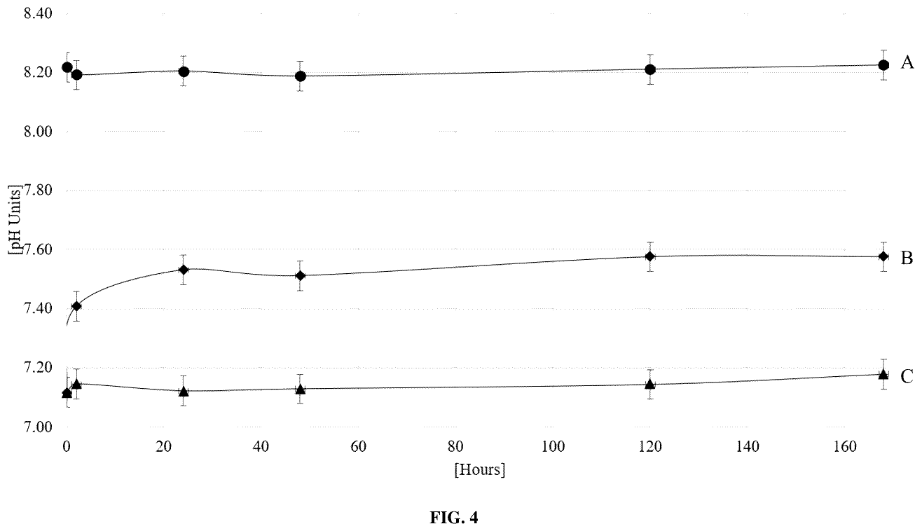

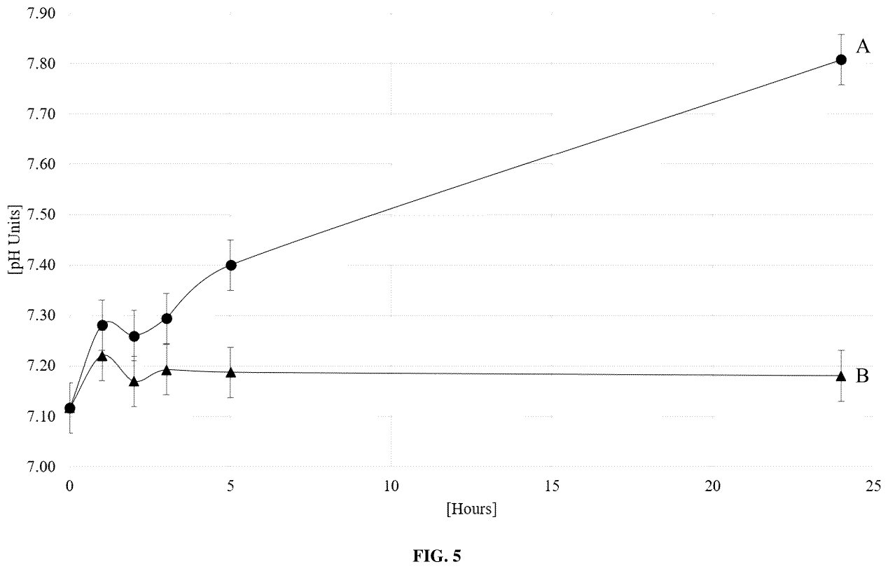

Finally, the pH of the 12.6 mg/mL sodium bicarbonate solution of Table 1 was evaluated using overwrapped (FIG. 4) and not overwrapped (FIG. 5) Renolit Solmed Infuflex 9101 infusion bags with and without an initial pH adjustment to pH 7.0 using carbon dioxide and/or carbon dioxide headspace (HS) and with and without an infusion set. The results show that the pH is stable for the ready-to-use 12.6 mg/mL sodium bicarbonate solution contained in Renolit Solmed Infuflex 9101 infusion bag as primary packaging and without headspace (i.e., with less than 2% of the total infusion bag volume as headspace). Additionally, the presence of the infusion set does not affect the formation of carbon dioxide and the pH of the solution remains stable in the presence of the infusion set for the sodium bicarbonate product of the present disclosure contained in Renolit Solmed Infuflex 9101 infusion bag without headspace. The results are shown in FIGS. 4-5.

The inventors also observed carbon dioxide bubble formation in three replicates of the ready-to- 12.6 mg/mL sodium bicarbonate solution of Table 1 in a Renolit Solmed Infuflex 9101 infusion bag without an overwrap immediately after preparation and 168 hours after preparation; bubble formation within the infusion set during the infusion process; bubble formation before and after shaking the bag; and bubble formation and pH in three different batches of the 12.6 mg/mL sodium bicarbonate solution of Table 1 in a Renolit Solmed Infuflex 9101 infusion bag during storage and after one hour of infusion. The tests show that no significant bubble formation was detected and that bubble formation was further reduced when an overwrap is present.

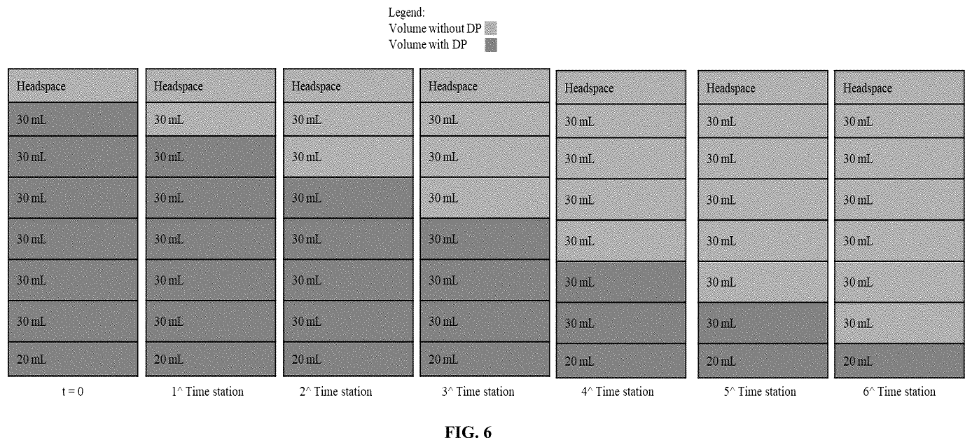

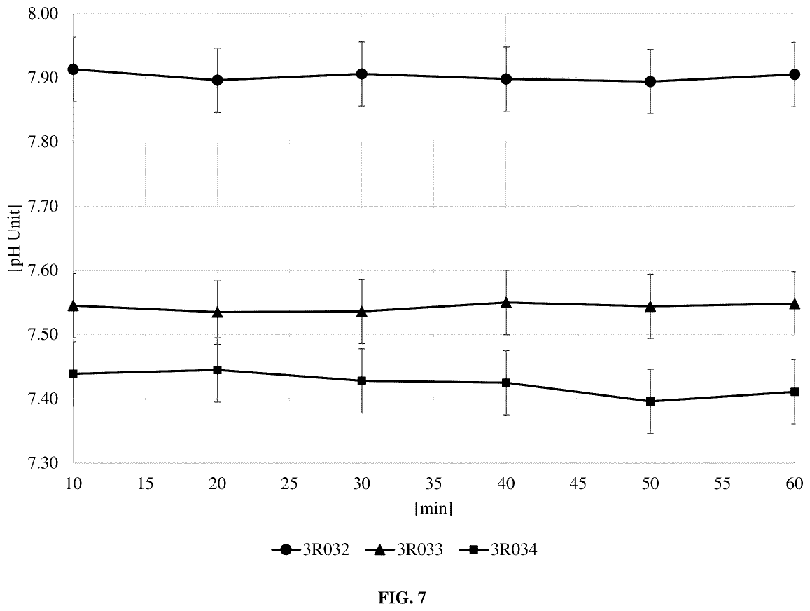

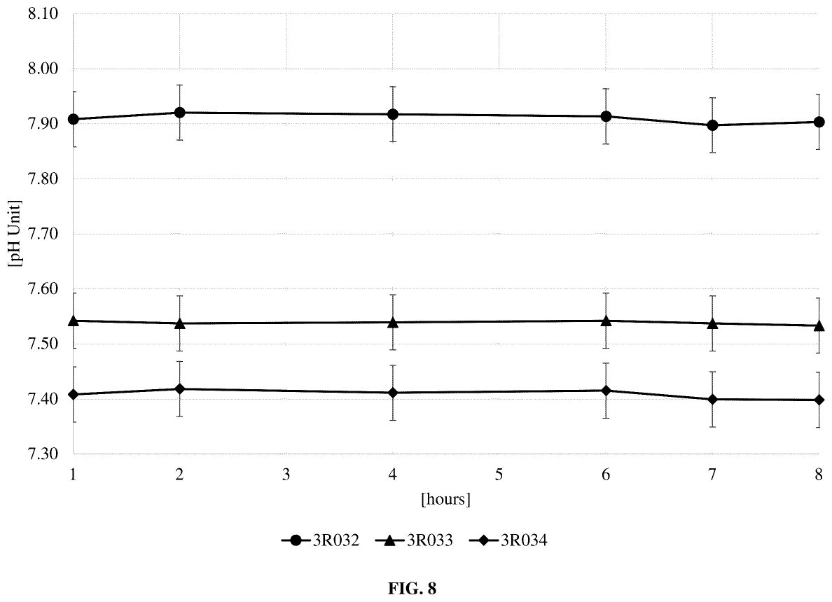

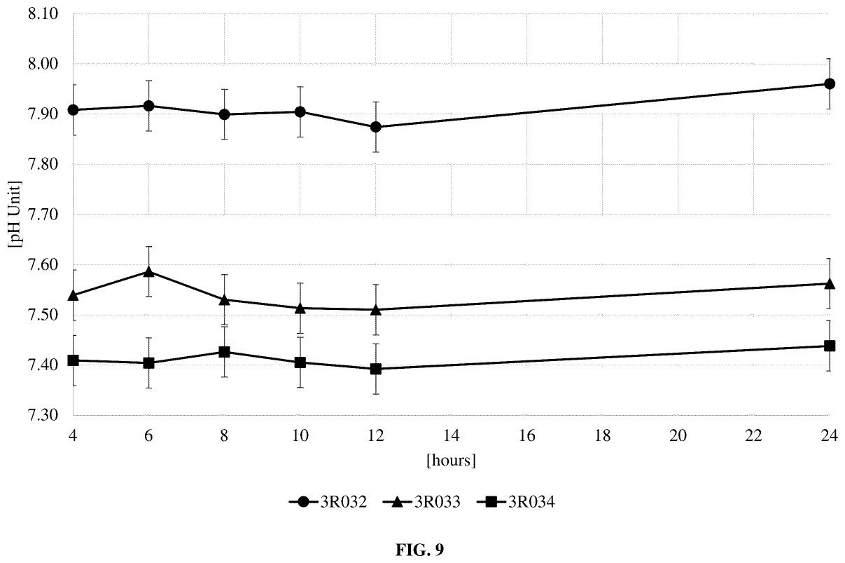

Finally, the inventors tested the change of pH at 10 to 60 minutes of continuous infusion (FIG. 7), one hour to eight hours of continuous infusion (FIG. 8), 12 to 24 hours of continuous infusion (FIG. 9) in three batches of the ready-to-use 12.6 mg/mL sodium bicarbonate product of the present disclosure in a 200 mL Renolit Solmed Infuflex 9101 infusion bag. The three batches were batches 3R-032, 3R-033, and 3R-034, prepared as described above in Example 2. FIG. 6 shows the change of ratio of headspace volume to sodium bicarbonate solution volume during continuous infusion, wherein each time station of FIG. 6 is: 1\hat{ }Time station is 10 minutes of continuous infusion for a total of 1 hour of infusion time, 1 hour of infusion for a total of 8 hours of infusion time, and 4 hours of infusion for a total of 24 hours of infusion time; 2\hat{ }Time station is 20 minutes of continuous infusion for a total of 1 hour of infusion time, 2 hour of infusion for a total of 8 hours of infusion time, and 6 hours of infusion for a total of 24 hours of infusion time; 3\hat{ }Time station is 30 minutes of continuous infusion for a total of 1 hour of infusion time, 4 hours of infusion for a total of 8 hours of infusion time, and 8 hours of infusion for a total of 24 hours of infusion time; 4\hat{ }Time station is 40 minutes of continuous infusion for a total of 1 hour of infusion time, 6 hour of infusion for a total of 8 hours of infusion time, and 10 hours of infusion for a total of 24 hours of infusion time; 5\hat{ }Time station is 50 minutes of continuous infusion for a total of 1 hour of infusion time, 7 hours of infusion for a total of 8 hours of infusion time, and 12 hours of infusion for a total of 24 hours of infusion time; 6\hat{ }Time station is 60 minutes of continuous infusion for a total of 1 hour of infusion time, 8 hours of infusion for a total of 8 hours of infusion time, and 24 hours of infusion for a total of 24 hours of infusion time. According to Table 9 below:

TABLE 9
Time station for 1 to 24 hours of infusion
Time station scheduled for 1 hour of infusion

| | | | | | |
|---|---|---|---|---|---|
| 10 min | 20 min | 30 min | 40 min | 50 min | 60 min |

Time station scheduled for 4-8 hours of infusion

| | | | | | |
|---|---|---|---|---|---|
| 1 h | 2 h | 4 h | 6 h | 7 h | 8 h |

Time station scheduled for 12-24 hours of infusion

| | | | | | |
|---|---|---|---|---|---|
| 4 h | 6 h | 8 h | 10 h | 12 h | 24 h |

All three tested batches showed that the pH of the sodium bicarbonate product of the present disclosure in the Renolit Solmed Infuflux 9101 infusion bag is stable during the infusion time in all tested conditions. Additionally, the inventors surprisingly found the initial pH value and the ratio between headspace and sodium bicarbonate solution and the infusion rate (different infusion rate tested) has no influence on the pH stability during infusion time of the ready-to-use sodium bicarbonate product of the present disclosure in the Renolit Solmed Infuflux 9101 infusion bag.

### Example 4: CO2 Insufflation

The CO2 insufflation of the ready-to-use sodium bicarbonate product of the present disclosure during the manufacturing process described in Example 2 was evaluated at three different pH targets (7.8/7.5/7.2). The results are shown in Tables 10, 11, and 12 below.

TABLE 10
Dissolution and flushing results of Lot 3R032

| | | | Lot 3R032 (target pH = 7.8) | |
|---|---|---|---|---|
| | | | Dissolution | Flushing |
| | | | 10 min (from pH | After 60 kg |
| Test | Acceptance criteria | Units | target reaching) | Flushing |
| Clarity and | Clear and colourless | — | Comply | Comply |
| Color | solution | | | |
| Appearance | Free from visible | — | Comply | Comply |
| | particles | | | |
| Bicarbonate | 95.0-105.0 | % | 99.56 | 97.83 |
| Assay | | | | |
| pH | 7.0-8.5 | pH | 7.849 | 7.871 |
| | | Units | | |
| Osmolality | For information only | mOsmol/ | 536 | 534 |
| | | kg | | |
| Conductivity | For information only | mS/cm | 9.681 | 9.555 |
| Density | For information only | g/cm3 | 1.02277 | 1.02215 |

TABLE 11
Dissolution and flushing results of Lot 3R033

| | | | Lot 3R033 (target pH = 7.5) | |
|---|---|---|---|---|
| | | | Dissolution | |
| | | | 10 min (from | Flushing |
| | | | pH target | After 60 kg |
| Test | Acceptance criteria | Units | reaching) | Flushing |
| Clarity and Color | Clear and colourless solution | — | Comply | Comply |
| Appearance | Free from visible particles | — | Comply | Comply |
| Bicarbonate Assay | 95.0-105.0 | % | 97.71 | 97.66 |
| pH | 7.0-8.5 | pH Units | 7.459 | 7.426 |
| Osmolality | For information only | mOsmol/kg | 533 | 532 |
| Conductivity | For information only | mS/cm | 9.562 | 9.581 |
| Density | For information only | g/cm3 | 1.02256 | 1.02246 |

TABLE 12
Dissolution and flushing results of Lot 3R034

| | | | Lot 3R034 (target pH = 7.2) | |
|---|---|---|---|---|
| | | | Dissolution | |
| | | | 10 min (from | Flushing |
| | | | pH target | After 60 kg |
| Test | Acceptance criteria | Units | reaching) | Flushing |
| Clarity and Color | Clear and colourless solution | — | Comply | Comply |
| Appearance | Free from visible particles | — | Comply | Comply |
| Bicarbonate Assay | 95.0-105.0 | % | 97.97 | 97.87 |
| pH | 7.0-8.5 | pH Units | 7.309 | 7.287 |
| Osmolality | For information only | mOsmol/kg | 539 | 543 |
| Conductivity | For information only | mS/cm | 9.555 | 9.560 |
| Density | For information only | g/cm3 | 1.02252 | 1.02250 |

All three tested batches at each pH target (7.8/7.5/7.2) complied with the acceptance criteria for clarity and color, appearance, bicarbonate assay, pH, osmolarity, conductivity and density.

### Example 5: Tank Holding Time Study

The critical IPC variation of the sodium bicarbonate solution of the present disclosure was evaluated after contact with a stainless steel tank for 134 hours, after contact with a static filter for 84 hours, and after contact with a PFA tube for 8 hours.

No critical IPC variation was noted and the pH remained stable after contact with stainless steel tank for 134 hours and after contact with a static filter for 84 hours. No absorption of the sodium bicarbonate solution was observed after contact with a PFA tube for 8 hours, however some increase in pH was observed at 8 hours of contact. Therefore, the sodium bicarbonate solution of the disclosure should be dispensed into Renolit Solmed Infuflex 9101 infusion bags no later than 8 hours after contact with the PFA tube.

### Example 6: Stability Studies

The accelerated and long-term stability of six batches of the of the ready-to-use sodium bicarbonate product of the present disclosure at three pH targets (7.8/7.5/7.2) according to Table 13 below was evaluated. The batches were prepared as described in Example 2, aseptically filled through a 0.22 micron filter into Renolit Solmed 9101 bags, and overwrapped with either duplex or quadruplex overwrap film, wherein the quadruplex overwrap film comprises at least one layer of aluminum and the duplex overwrap does not contain aluminum. The overwrap was not vacuum sealed over the primary packaging.

TABLE 13
Stability Batches

| Product | Batch | pH target | Secondary packaging |
|---|---|---|---|
| Sodium Bicarbonate | 3R032-Q | 7.8 | Quadruplex |
| 12.6 mg/mL | 3R032-D | | Duplex (PP/PE) |
| | 3R033-Q | 7.5 | Quadruplex |
| | 3R033-D | | Duplex (PP/PE) |
| | 3R034-Q | 7.2 | Quadruplex |
| | 3R034-D | | Duplex (PP/PE) |

Each batch described in Table 13 above was tested at accelerated conditions of 40±2° C./<25% RH for 6 months. The results of each batch are shown in Tables 14, 15, 16, 17, 18 and 19 below.

TABLE 14
3R032-Q Accelerated Condition 40 ± 2° C./<25% RH - 6 months

| TEST | Shelf-life | T = 0 | 1 Month | 2 Months | 3 Months | 6 Months |
|---|---|---|---|---|---|---|
| Appearance: | | | | | | |
| Clear and | Complies | complies | complies | does not | does not | does not |
| colorless | | | | comply | comply | comply |
| solution | | | | | | |
| Free from | Complies | complies | complies | complies | complies | complies |
| visible | | | | | | |
| particles | | | | | | |
| Bicarbonate | 95.0- | 97.9 | 95.9 | 93.0 | 91.0 | 88.9 |
| Assay (as | 105.0% | | | | | |
| nominal) | | | | | | |
| pH | 7.0-8.5 pH | 7.8 | 7.8 | 7.7 | 7.6 | 7.5 |
| | Units | | | | | |
| Osmolality | 500-580 | 529 | 520 | 521 | 526 | 519 |
| | mOsmolKg | | | | | |
| Particulate | | | | | | |
| Matter: | | | | | | |
| Part. ≥10 | ≤25 | 2 | 0 | — | 0 | 4 |
| micron | Nr. PartmL | | | | | |
| Part. ≥25 | ≤3 | 0 | 0 | — | 0 | 1 |
| micron | Nr. PartmL | | | | | |

TABLE 15
3R033-Q Accelerated Condition 40 ± 2° C./<25% RH - 6 months

| TEST | shelflife | T = 0 | 1 Month | 2 Months | 3 Months | 6 Months |
|---|---|---|---|---|---|---|
| Appearance: | | | | | | |
| Clear and | Complies | complies | complies | complies | complies | does not |
| colorless | | | | | | comply |
| solution | | | | | | |
| Free from | Complies | complies | complies | complies | complies | complies |
| visible | | | | | | |
| particles | | | | | | |
| Bicarbonate | 95.0- | 98.0 | 96.5 | 93.9 | 91.8 | 89.5 |
| Assay (as | 105.0% | | | | | |
| nominal) | | | | | | |
| pH | 7.0-8.5 | 7.4 | 7.7 | 7.6 | 7.5 | 7.4 |
| | pH Units | | | | | |
| Osmolality | 500-580 | 534 | 525 | 526 | 531 | 524 |
| | mOsmolKg | | | | | |
| Particulate | | | | | | |
| Matter: | | | | | | |
| Part. ≥10 | ≤25 | 1 | 0 | — | 0 | 1 |
| micron | Nr. PartmL | | | | | |
| Part. ≥25 | ≤3 | 0 | 0 | — | 0 | 0 |
| micron | Nr. PartmL | | | | | |

TABLE 16
3R034-Q Accelerated Condition 40 ± 2° C./<25% RH - 6 months

| TEST | shelflife | T = 0 | 1 Month | 2 Months | 3 Months | 6 months |
|---|---|---|---|---|---|---|
| Appearance: | | | | | | |
| Clear and | Complies | Complies | Complies | Complies | Complies | Does not |
| colorless | | | | | | comply |
| solution | | | | | | |
| Free from | Complies | Complies | Complies | Complies | Complies | Complies |
| visible | | | | | | |
| particles | | | | | | |
| Bicarbonate | 95.0- | 97.9 | 96.4 | 94.1 | 92.1 | 89.6 |
| Assay (as | 105.0% | | | | | |
| nominal) | | | | | | |
| pH | 7.0-8.5 pH | 7.3 | 7.6 | 7.6 | 7.5 | 7.4 |
| | Units | | | | | |
| Osmolality | 500-580 | 539 | 531 | 528 | 530 | 507 |
| | mOsmolKg | | | | | |
| Particulate | | | | | | |
| Matter: | | | | | | |
| Part. ≥10 | ≤25 | 1 | 1 | — | 0 | 1 |
| micron | Nr. PartmL | | | | | |
| Part. ≥25 | ≤3 | 0 | 0 | — | 0 | 0 |
| micron | Nr. PartmL | | | | | |

TABLE 17
3R032-D Accelerated Condition 40 ± 2° C./<25% RH - 6 months

| TEST | shelflife | T = 0 | 1 Month | 2 Months | 3 Months | 6 months |
|---|---|---|---|---|---|---|
| Appearance: | | | | | | |
| Clear and | Complies | complies | does not | does not | does not | does not |
| colorless | | | comply | comply | comply | comply |
| solution | | | | | | |
| Free from | Complies | complies | complies | complies | complies | complies |
| visible | | | | | | |
| particles | | | | | | |
| Bicarbonate | 95.0- | 97.9 | 98.0 | 98.5 | 98.9 | 98.5 |
| Assay (as | 105.0% | | | | | |
| nominal) | | | | | | |
| pH | 7.0-8.5 | 7.8 | 8.0 | 8.1 | 8.2 | 8.1 |
| | pH Units | | | | | |
| Osmolality | 500-580 | 529 | 528 | 526 | 531 | 532 |
| | mOsmolKg | | | | | |
| Particulate | | | | | | |
| Matter: | | | | | | |
| Part. ≥10 | ≤25 | 2 | 1 | — | 1 | 2 |
| micron | Nr. PartmL | | | | | |
| Part. ≥25 | ≤3 | 0 | 0 | — | 0 | 1 |
| micron | Nr. PartmL | | | | | |

TABLE 18
3R033-D Accelerated Condition 40 ± 2° C./<25% RH - 6 months

| TEST | shelflife | T = 0 | 1 Month | 2 Months | 3 Months | 6 months |
|---|---|---|---|---|---|---|
| Appearance: | | | | | | |
| Clear and | Complies | complies | does not | does not | does not | does not |
| colorless | | | comply | comply | comply | comply |
| solution | | | | | | |
| Free from | Complies | complies | complies | complies | complies | complies |
| visible | | | | | | |
| particles | | | | | | |
| Bicarbonate | 95.0- | 98.0 | 97.8 | 98.1 | 97.4 | 97.4 |
| Assay (as | 105.0% | | | | | |
| nominal) | | | | | | |
| pH | 7.0-8.5 | 7.4 | 7.8 | 7.9 | 8.2 | 8.2 |
| | pH Units | | | | | |
| Osmolality | 500-580 | 534 | 528 | 522 | 542 | 537 |
| | mOsmolKg | | | | | |
| Particulate | | | | | | |
| Matter: | | | | | | |
| Part. ≥10 | ≤25 | 1 | 1 | — | 1 | 2 |
| micron | Nr. PartmL | | | | | |
| Part. ≥25 | ≤3 | 0 | 0 | — | 0 | 1 |
| micron | Nr. PartmL | | | | | |

TABLE 19
3R034-D Accelerated Condition 40 ± 2° C./<25% RH - 6 months

| TEST | shelflife | T = 0 | 1 Month | 2 Months | 3 Months | 6 months |
|---|---|---|---|---|---|---|
| Appearance: | | | | | | |
| Clear and | Complies | complies | complies | does not | does not | does not |
| colorless | | | | comply | comply | comply |
| solution | | | | | | |
| Free from | Complies | complies | complies | complies | complies | complies |
| visible | | | | | | |
| particles | | | | | | |
| Bicarbonate | 95.0- | 97.9 | 97.7 | 97.5 | 98.0 | 96.9 |
| Assay (as | 105.0% | | | | | |
| nominal) | | | | | | |
| pH | 7.0-8.5 | 7.3 | 7.7 | 8.0 | 8.1 | 8.2 |
| | pH Units | | | | | |
| Osmolality | 500-580 | 539 | 532 | 532 | 531 | 535 |
| | mOsmolKg | | | | | |
| Particulate | | | | | | |
| Matter: | | | | | | |
| Part. ≥10 | ≤25 | 1 | 1 | — | 1 | 2 |
| micron | Nr. PartmL | | | | | |
| Part. ≥25 | ≤3 | 0 | 0 | — | 0 | 0 |
| micron | Nr. PartmL | | | | | |

Each batch described in Table 13 above was also tested at long term conditions of 25±2° C./40±5% RH for 9 months. The results of each batch are shown in Tables 20, 21, 22, 23, 24 and 25 below

TABLE 20
3R034-D Long Term Condition 25 ± 2° C./40 ± 5% RH - 9 months

| TEST | Shelf-life | T = 0 | 1 Month | 2 Months | 3 Months | 6 months | 9 Months |
|---|---|---|---|---|---|---|---|
| Appearance: | | | | | | | |
| Clear and | Complies | complies | complies | complies | complies | complies | complies |
| colorless | | | | | | | |
| solution | | | | | | | |
| Free from | Complies | complies | complies | complies | complies | complies | complies |
| visible | | | | | | | |
| particles | | | | | | | |
| Bicarbonate | 95.0- | 97.9 | 97.9 | 97.9 | 97.9 | 97.6 | 97.3 |
| Assay (as | 105.0% | | | | | | |
| nominal) | | | | | | | |
| pH | 7.0-8.5 | 7.3 | 7.5 | 7.6 | 7.7 | 7.9 | 8.1 |
| | pH Units | | | | | | |
| Osmolality | 500-580 | 539 | 534 | 526 | 535 | 527 | 530 |
| | mOsmolKg | | | | | | |
| Particulate | | | | | | | |
| Matter: | | | | | | | |
| Part. ≥10 | ≤25 | 1 | — | — | — | 4 | — |
| micron | Nr. PartmL | | | | | | |
| Part. ≥25 | ≤3 | 0 | — | — | — | 1 | — |
| micron | Nr. PartmL | | | | | | |

TABLE 21
3R032-Q Long Term Condition 25 ± 2° C./40 ± 5% RH - 9 months

| TEST | Shelf-life | T = 0 | 1 Month | 2 Months | 3 Months | 6 months | 9 Months |
|---|---|---|---|---|---|---|---|
| Appearance: | | | | | | | |
| Clear and | Complies | complies | complies | complies | complies | complies | complies |
| colorless | | | | | | | |
| solution | | | | | | | |
| Free from | Complies | complies | complies | complies | complies | complies | complies |
| visible | | | | | | | |
| particles | | | | | | | |
| Bicarbonate | 95.0- | 97.9 | 97.8 | 97.5 | 97.0 | 96.6 | 95.4 |
| Assay (as | 105.0% | | | | | | |
| nominal) | | | | | | | |
| pH | 7.0-8.5 | 7.8 | 8.1 | 8.1 | 8.0 | 7.9 | 7.8 |
| | pH Units | | | | | | |
| Osmolality | 500-580 | 529 | 518 | 520 | 528 | 521 | 526 |
| | mOsmolKg | | | | | | |
| Particulate | | | | | | | |
| Matter: | | | | | | | |
| Part. ≥10 | ≤25 | 2 | — | — | — | 2 | — |
| micron | Nr. PartmL | | | | | | |
| Part. ≥25 | ≤3 | 0 | — | — | — | 1 | — |
| micron | Nr. PartmL | | | | | | |

TABLE 22
3R033-Q Long Term Condition 25 ± 2° C./40 ± 5% RH - 9 months

| TEST | Shelf-life | T = 0 | 1 Month | 2 Months | 3 Months | 6 months | 9 Months |
|---|---|---|---|---|---|---|---|
| Appearance: | | | | | | | |
| Clear and | Complies | complies | complies | complies | complies | complies | complies |
| colorless | | | | | | | |
| solution | | | | | | | |
| Free from | Complies | complies | complies | complies | complies | complies | complies |
| visible | | | | | | | |
| particles | | | | | | | |
| Bicarbonate | 95.0- | 98.0 | 97.9 | 97.8 | 97.6 | 96.9 | 96.1 |
| Assay (as | 105.0% | | | | | | |
| nominal) | | | | | | | |
| pH | 7.0-8.5 | 7.4 | 7.8 | 7.8 | 7.8 | 7.7 | 7.7 |
| | pH Units | | | | | | |
| Osmolality | 500-580 | 534 | 524 | 524 | 529 | 525 | 527 |
| | mOsmolKg | | | | | | |
| Particulate | | | | | | | |
| Matter: | | | | | | | |
| Part. ≥10 | ≤25 | 1 | — | — | — | 3 | — |
| micron | Nr. PartmL | | | | | | |
| Part. ≥25 | ≤3 | 0 | — | — | — | 0 | — |
| micron | Nr. PartmL | | | | | | |

TABLE 23
3R034-Q Long Term Condition 25 ± 2° C./40 ± 5% RH - 9 months

| TEST | Shelf-life | T = 0 | 1 Month | 2 Months | 3 Months | 6 months | 9 Months |
|---|---|---|---|---|---|---|---|
| Appearance: | | | | | | | |
| Clear and | Complies | complies | complies | complies | complies | complies | complies |
| colorless | | | | | | | |
| solution | | | | | | | |
| Free from | Complies | complies | complies | complies | complies | complies | complies |
| visible | | | | | | | |
| particles | | | | | | | |
| Bicarbonate | 95.0- | 97.9 | 97.9 | 97.7 | 97.5 | 96.9 | 96.3 |
| Assay (as | 105.0% | | | | | | |
| nominal) | | | | | | | |
| pH | 7.0-8.5 pH | 7.3 | 7.7 | 7.6 | 7.6 | 7.6 | 7.6 |
| | Units | | | | | | |
| Osmolality | 500-580 | 539 | 530 | 522 | 530 | 524 | 529 |
| | mOsmolKg | | | | | | |
| Particulate | | | | | | | |
| Matter: | | | | | | | |
| Part. ≥10 | ≤25 | 1 | — | — | — | 2 | — |
| micron | Nr. PartmL | | | | | | |
| Part. ≥25 | ≤3 | 0 | — | — | — | 0 | — |
| micron | Nr. PartmL | | | | | | |

TABLE 24
3R032-D Long Term Condition 25 ± 2° C./40 ± 5% RH - 9 months

| TEST | Shelf-life | T = 0 | 1 Month | 2 Months | 3 Months | 6 months | 9 Months |
|---|---|---|---|---|---|---|---|
| Appearance: | | | | | | | |
| Clear and | Complies | complies | complies | complies | complies | complies | complies |
| colorless | | | | | | | |
| solution | | | | | | | |
| Free from | Complies | complies | complies | complies | complies | complies | complies |
| visible | | | | | | | |
| particles | | | | | | | |
| Bicarbonate | 95.0- | 97.9 | 97.6 | 97.7 | 98.0 | 97.8 | 97.5 |
| Assay (as | 105.0% | | | | | | |
| nominal) | | | | | | | |
| pH | 7.0-8.5 pH | 7.8 | 7.9 | 7.9 | 8.0 | 8.1 | 8.2 |
| | Units | | | | | | |
| Osmolality | 500-580 | 529 | 522 | 521 | 527 | 525 | 528 |
| | mOsmolKg | | | | | | |
| Particulate | | | | | | | |
| Matter: | | | | | | | |
| Part. ≥10 | ≤25 | 2 | — | — | — | 2 | — |
| micron | Nr. PartmL | | | | | | |
| Part. ≥25 | ≤3 | 0 | — | — | — | 1 | — |
| micron | Nr. PartmL | | | | | | |

TABLE 25
3R033-D Long Term Condition 25 ± 2° C./40 ± 5% RH - 9 months

| TEST | shelflife | T = 0 | 1 Month | 2 Months | 3 Months | 6 months | 9 Months |
|---|---|---|---|---|---|---|---|
| Appearance: | | | | | | | |
| Clear and | Complies | complies | complies | complies | complies | complies | complies |
| colorless | | | | | | | |
| solution | | | | | | | |
| Free from | Complies | complies | complies | complies | complies | complies | complies |
| visible | | | | | | | |
| particles | | | | | | | |
| Bicarbonate | 95.0- | 98.0 | 98.1 | 97.7 | 97.8 | 97.5 | 97.2 |
| Assay (as | 105.0% | | | | | | |
| nominal) | | | | | | | |
| pH | 7.0-8.5 pH | 7.4 | 7.6 | 7.7 | 7.8 | 8.0 | 8.1 |
| | Units | | | | | | |
| Osmolality | 500-580 | 534 | 527 | 523 | 529 | 525 | 527 |
| | mOsmolKg | | | | | | |
| Particulate | | | | | | | |
| Matter: | | | | | | | |
| Part. ≥10 | ≤25 | 1 | — | — | — | 2 | — |
| micron | Nr. PartmL | | | | | | |
| Part. ≥25 | ≤3 | 0 | — | — | — | 0 | — |
| micron | Nr. PartmL | | | | | | |

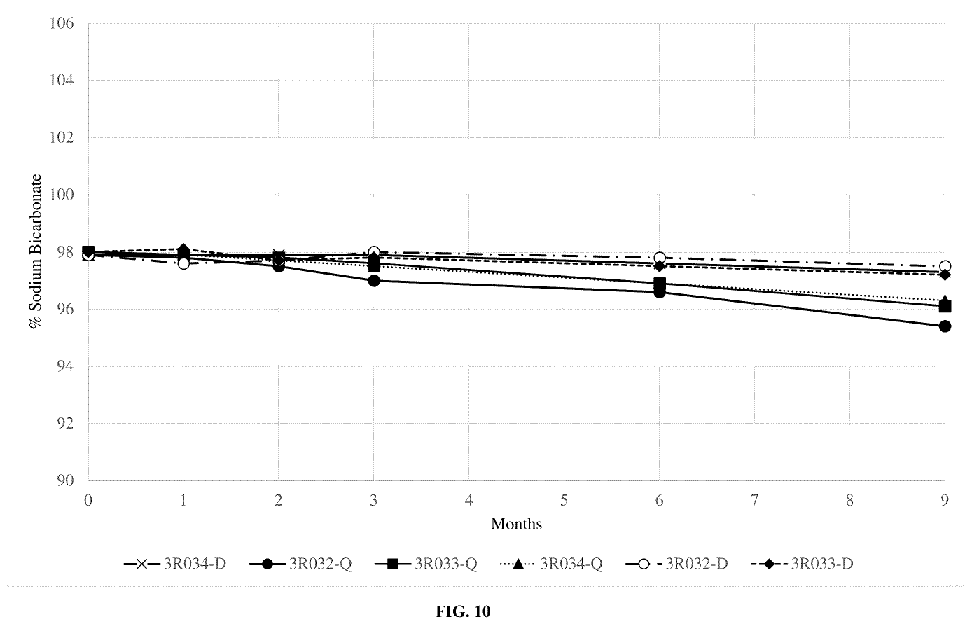

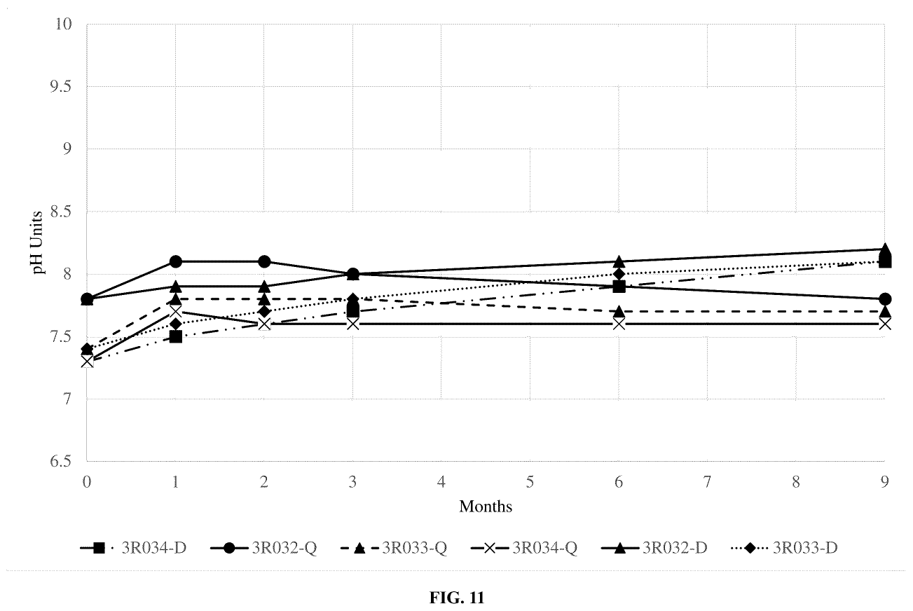

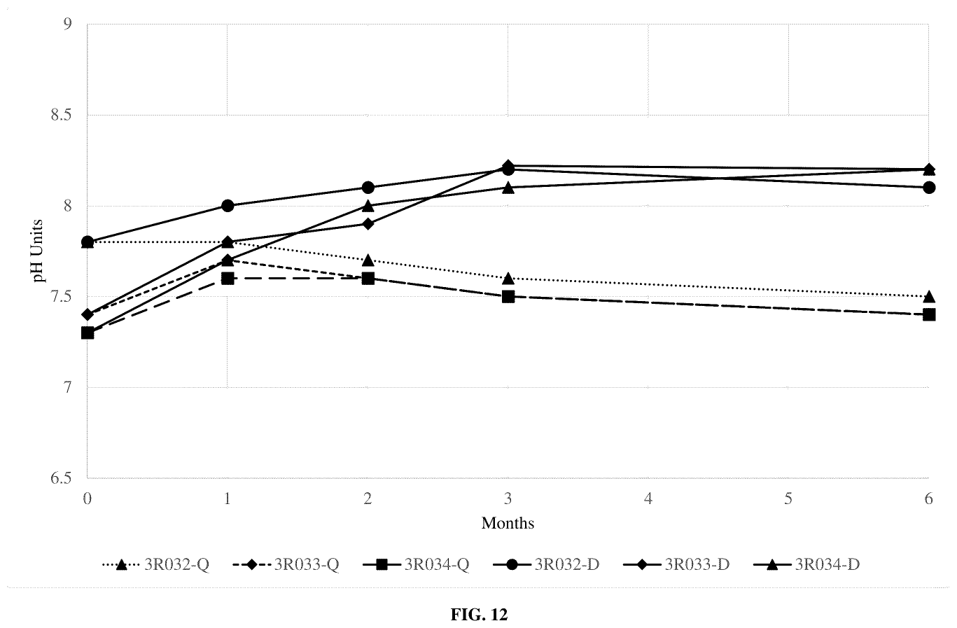

As shown in FIG. 12 and Tables 14-19, the ready-to-use sodium bicarbonate product of the present disclosure is highly sensitive to high temperatures of 40° C. However, the ready-to-use, aseptically filled sodium bicarbonate product of the present disclosure is stable after 9 months at 25° C. As shown in FIGS. 10 and 11, there was no observed reduction in bicarbonate assay compared to T=0 in the long term stability study. The visible particles, pH, osmolarity and particulate matter were also comparable with the results obtained at T=0.

The results of Tables 20-25 surprisingly show that the ready-to-use sodium bicarbonate product of the present disclosure, formulated without any antimicrobial or preservative, retains an average pH of 7-8.5 pH units and a sodium bicarbonate assay of 95-105% of the nominal value when stored at room temperature for prolonged periods. The results also show that using a quadruplex overwrap comprising at least one layer of aluminum reduces the change in pH of the ready-to-use sodium bicarbonate product of the present disclosure over time compared to using a duplex overwrap, which contains no aluminum, if there is some headspace remaining in the infusion bag. Additionally, the osmolality of the ready-to-use, aseptically filled sodium bicarbonate product of the present disclosure remained inside the acceptance criteria of 500-580 mOsmol/kg during long-term stability studies. No significant change was noted during the long-term stability studies and no degradation was detected despite of a lack of any antimicrobial or preservative in the ready-to-use sodium bicarbonate product of the present disclosure.

### Example 7: Degradation Study in Glass Vial.

Batches of the sodium bicarbonate solution of the disclosure were packaged in a glass vial (“DP”) and studied for 7 days at storage temperatures of 80° C. with and without headspace and air exposure according to Table 26 and 27 below:

TABLE 26
First Experimental Plan for Degradation Analysis

| | | Condition | Condition |
|---|---|---|---|
| Sample | Description | 1 | 2 |
| Dextrose solution | 50 mg/mL | Thermal | Thermal + |
| | Dextrose | stress | air exposure |
| Sodium Bicarbonate | 12.6 mg/mL | GV without | stress |
| | NaHCO3 | HS 7 days | GV with HS |
| Drug Product | DP Fresh | at 80° C. | (ratio |
| without CO2 | Formulation | | Sample: |
| Drug Product pH 7.8 | Batch 3R032Q | | HS = 1:6) |
| Drug Product pH 7.5 | Batch 3R033Q | | 7 days at |
| Drug Product pH 7.2 | Batch 3R034Q | | 80° C. |

TABLE 27
Second Experimental Plan for Degradation Analysis

| Sample | Description | Stress Condition |
|---|---|---|
| Drug Product pH 7.8 | Batch 3R032Q | Thermal stress |
| Drug Product pH 7.5 | Batch 3R033Q | GV without HS |
| Drug Product pH 7.2 | Batch 3R034Q | 2 days at 80° C. |

The batches of the DP were analyzed with the UV-VIS at wavelength 284 nm to detect 5-hydroxymethylfurfural (5-HMF) formation. The results are shown in Table 28 below:

TABLE 28
5 HMF quantification by UV-VIS method

| Sample name* | Avg Conc [mg/mL] | 5HMF conc [%] |
|---|---|---|
| Dextrose no CO2 no HS | 0.007 | 0015 |
| Dextrose no CO2 HS | 0.026 | 0.052 |
| DP no CO2 no HS | 0.320 | 0.640 |
| DP no CO2 HS | 0.706 | 1.413 |
| 3R032 + CO2 no HS | 0.510 | 1.019 |
| 3R032 + CO2 HS | 0.514 | 1.028 |
| 3R034 + CO2 no HS | 0.467 | 0.934 |
| 3R034 + CO2 HS | 0.818 | 1.636 |

Surprisingly, the results indicate that the intense coloration of the stressed samples (DP, 3R032, 3R033 and 3R034) could not be explained by 5-HMF formation during thermal degradation.

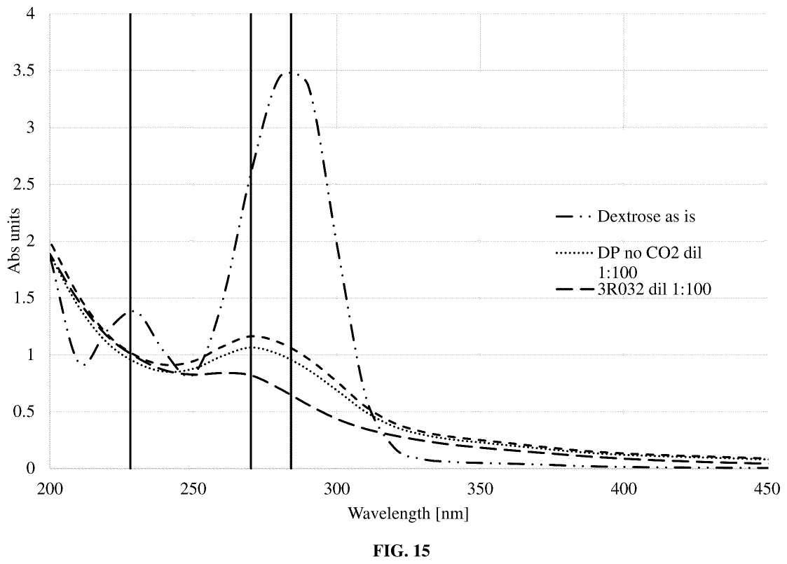

A second UV-VIS analysis was performed at wavelength 200-800 nm. DS lots 3R032 and 3R034 packaged without headspace were diluted 1:100 due to reduce intense coloration in order to obtain an Abs<3 Abs Units. Only the stressed samples without HS have been analyzed by Scan mode. The results are shown in FIG. 15.

The dextrose stressed sample spectrum shows two evident maxima of absorbance at 284 nm due to 5-HMF formation during thermal degradation and at 228 nm, probably due to the 3,4-Dideoxyglucosone-3-ene formation. The DP stressed samples (DP no CO₂, 3R032 and 3R034) have a different spectrum of absorbance than that of dextrose (maximum at 270 nm). This result indicates that, due to the intense coloring of those samples, the first UV-VIS analysis of those samples were not reliable. The stressed dextrose sample, with a much higher concentration of 5-HMF than the stressed DP samples, is clear and colorless. The stressed DPs, even diluted 1:100, are characterized by a yellow-brown color.

Additionally, the inventors surprisingly found that the presence of HS accelerates the degradation of the sample solution. The inventors found that visible coloration only presents in glass vials containing both sodium bicarbonate and dextrose. The intensity of color of the solution appears to be inversely proportional to the amount of carbon dioxide present in the solution. This aspect is not appreciable in conditions with HS as the coloring becomes too intense in all samples to qualify the color difference. 3R033 no HS unexpectedly shows coloration. The same sample placed in different positions in the muffle show different degrees of degradation, this is likely due to the small temperature differences in the different positions of the muffle.

The pH of the stressed samples according to the conditions described in Table 26 above are shown in Table 29 below:

TABLE 29
pH Results of Stressed Samples

| | Sample | | | | | |
|---|---|---|---|---|---|---|
| Conditions | NaHCO3 | Dextrose | DP no CO2 | 3R032 | 3R033 | 3R034 |
| No HS | 8.7 | 4.6 | 7.8 | 7.4 | 7.5 | 7.2 |
| HS | 9.0 | 4.4 | 7.1 | 5.3 | 5.5 | 7.1 |

The increase in pH in the NaHCO3sample is due to a conversion of bicarbonate to carbonate according to the following thermal degradation reaction: 2NaHCO3→Na2CO3+H2O+CO2. The change in pH between the two conditions tested (HS/No HS) is not constant for all of the stressed samples.

In summary, the inventors found that high temperatures caused degradation of sodium bicarbonate and significant change in color. The degradation is due to the combination of dextrose and sodium bicarbonate in solution because the individual components subjected to the same stress conditions do not develop any coloration. Furthermore, the coloration in samples stable at 40° C. is not due to the formation of 5-HMF. Therefore, the present disclosure includes producing a sterile product for parenteral use using aseptic conditions rather than through terminal sterilization.

It is to be understood that any particular aspect of the present disclosure that falls within the prior art may be excluded from any one or more of the claims. Since such aspects are deemed to be part of the whole of the present disclosure, any part of the whole disclosure may be excluded even if the exclusion is not set forth explicitly herein. Additionally, it is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

1. A sodium bicarbonate product comprising an aqueous sodium bicarbonate solution comprising sodium bicarbonate at a concentration of about 0.1 mEq/mL to about 0.2 mEq/mL, a tonicity agent, and water; wherein the sodium bicarbonate product is sterile and ready-to-use; wherein the sodium bicarbonate solution is contained in an infusion bag comprising a flexible, multilayer film with at least one layer comprising maleic anhydride grafted polyethylene and at least one layer comprising poly(ethylene-co-1-octene); and wherein the pH of the aqueous sodium bicarbonate solution is maintained within a range of about 7.0 to 8.5 following storage for 6 months at a controlled room temperature of 25° C.±2° C. with relative humidity (RH) at 40%±5%.

2. The sodium bicarbonate product of claim 1, wherein the concentration of sodium bicarbonate is about 0.1 mEq/mL or about 0.15 mEq/mL.

3. The sodium bicarbonate product of claim 1, wherein the carbon dioxide permeability of the infusion bag is less than 0.2 cm3/m2·day·bar.

4. The sodium bicarbonate product of claim 1, wherein the multilayer film comprises an outer layer, a middle layer, and a fluid contact layer, wherein one or more layers comprises maleic anhydride grafted polyethylene, and wherein the fluid contact layer comprises poly(ethylene-co-1-octene).

5. (canceled)

6. The sodium bicarbonate product of claim 1, wherein the multilayer film does not contain silicon oxide.

7. The sodium bicarbonate product of claim 1, wherein less than 5% of the total fill volume of the infusion bag comprises headspace.

8. The sodium bicarbonate product of claim 1, wherein the sodium bicarbonate product further comprises an overwrap surrounding the infusion bag.

9. The sodium bicarbonate product of claim 8, wherein the overwrap comprises aluminum and is not vacuum-sealed, and wherein the product comprises air between the infusion bag and the overwrap.

10. The sodium bicarbonate product of claim 1, wherein the pH of the sodium bicarbonate solution is maintained within a range of about 7.0 to 8.5 following storage for 12 months at a controlled room temperature of 25° C.±2° C. with relative humidity (RH) at 40%±5%.

11. The sodium bicarbonate product of claim 1, wherein the pH of the sodium bicarbonate solution is about 7.8 to 8.5.

12. (canceled)

13. The sodium bicarbonate product of claim 1, wherein the pH is maintained within a range of 7.5 to 8.5 following storage for 12 months at a controlled room temperature of 25° C.±2° C. with relative humidity (RH) at 40%±5%.

14. The sodium bicarbonate product of claim 1, wherein the buffering capacity (β) of the sodium bicarbonate solution is greater than 0.017.

15. (canceled)

16. (canceled)

17. The sodium bicarbonate product of claim 1, wherein the sodium bicarbonate solution is aseptically filled into the infusion bag.

18. A sodium bicarbonate product comprising:
(a) an aqueous sodium bicarbonate solution comprising about 0.1 to 0.2 mEq/mL sodium bicarbonate, a tonicity agent, and water for injection, and
(b) an infusion container comprising a flexible, multilayer film comprising an outer layer and at least one inner layer, wherein at least one inner layer comprises a polymer selected from the group consisting of poly(ethylene-co-1-octene) and maleic anhydride grafted polyethylene, and wherein the outer layer of the multilayer film does not comprise maleic anhydride grafted polyethylene;
wherein less than 5% of the total fill volume of the infusion container of (b) comprises headspace and the remainder of the fill volume comprises the aqueous sodium bicarbonate solution of (a); and
wherein the sodium bicarbonate product is sterile, ready-to-use and has a shelf life of the pH of the aqueous sodium bicarbonate solution is maintained within a range of about 7.0 to 8.5 for at least 6 months when the sodium bicarbonate product is stored at a controlled room temperature of 25° C.±2° C. with relative humidity (RH) at 40%±5%.

19. The sodium bicarbonate product of claim 18, wherein the sodium bicarbonate solution is aseptically filled into the infusion container.

20. The sodium bicarbonate product of claim 18, wherein the infusion container is an intravenous infusion bag, and wherein the multilayer film comprises an outer layer, a middle layer, and a fluid contact layer, wherein one or more layers comprises maleic anhydride grafted polyethylene, and wherein the fluid contact layer comprises poly(ethylene-co-1-octene).

21. The sodium bicarbonate product of claim 18, wherein the sodium bicarbonate is at a concentration of about 0.1 mEq/mL.

22. The sodium bicarbonate product of claim 18, wherein the sodium bicarbonate is at a concentration of about 0.15 mEq/mL.

23. The sodium bicarbonate product of claim 18, wherein the tonicity agent is dextrose monohydrate at a concentration of about 50 mg/mL.

24. The sodium bicarbonate product of claim 18, wherein the tonicity agent is sodium chloride at a concentration of about 9 mg/mL.

25. The sodium bicarbonate product of claim 18, wherein the sodium bicarbonate solution is free of antioxidants.

26. The sodium bicarbonate product of claim 18, wherein the pH of the sodium bicarbonate solution is about 7.8 to 8.5.

27. The sodium bicarbonate product of claim 18, wherein the pH of the sodium bicarbonate solution is maintained within a range of 7.5 to 8.5 following storage for 6 months at a controlled room temperature of 25° C.±2° C. with relative humidity (RH) at 40%±5%.

28. The sodium bicarbonate product of claim 18, wherein the sodium bicarbonate product further comprises an overwrap, wherein the overwrap comprises aluminum and is not vacuum-sealed, and wherein the product comprises air between the infusion container and the overwrap.

29. (canceled)

30. (canceled)

31. The sodium bicarbonate product of claim 18, wherein the sodium bicarbonate solution is free of preservatives.

32. The sodium bicarbonate product of claim 18, wherein the sodium bicarbonate solution is free of antimicrobials.
